(19) United States
(12) Patent Application Publication
(10) Pub. No.: US 2008/0004510 A1
(43) Pub. Date: Jan. 3, 2008

(54) Worn type electronic device and biological measuring apparatus provided with the same

(75) Inventors: Hiroki Tanzawa, Higashimurayama-shi (JP); Toshiyuki Kawakami, Kawagoe-shi (JP); Nobuto Fukushima, Sayama-shi (JP); Hideki Shimizu, Hanno-shi (JP); Tsuneharu Kasai, Tokorozawa-shi (JP); Shigeru Futakami, Tokorozawa-shi (JP); Kouji Fujii, Tokorozawa-shi (JP)

Correspondence Address: THE WEBB LAW FIRM, P.C., 700 KOPPERS BUILDING, 436 SEVENTH AVENUE, PITTSBURGH, PA, 15219, US

(73) Assignee: Citizen Watch Co., Ltd., Nishitokyo-shi (JP)

(21) Appl. No.: 11/716,753

(22) Filed: Mar. 12, 2007

(30) Foreign Application Priority Data
Mar. 13, 2006 (JP) 2006-067376
Mar. 14, 2006 (JP) 2006-069233
Feb. 13, 2007 (JP) 2007-032296

(51) Int. Cl. A61B 5/00

(52) U.S. Cl. 600/301

(57) ABSTRACT

A worn type electronic device comprises: a light emitting element for emitting a light having a specified wave length and a light guide means for guiding the light emitted from the light emitting element in a specified direction; the light emitting element disposed in a body of the worn type electronic device, wherein a light emission of the light emitting element is controlled based on the control of the worn type electronic device; and the light guide means including at least one of a body side light guide means disposed in the body of the worn type electronic device and a band side light guide means disposed in a band, wherein the light emitted from the light emitting element is emitted from the surface of the body of the worn type electronic device via the body side light guide means; or wherein the light emitted from the light emitting element is emitted from the surface of the band via the band side light guide means.

### TECHNICAL FIELD

The present invention relates to a worn type electronic device provided with a light emitting means for emitting a light from the worn type electronic device, such as a worn type electronic device configured in such a manner that a band or a body that configures an electronic wrist watch or the like is provided with a light emitting means, and a biological measuring apparatus provided with the worn type electronic device.

Moreover, the present invention relates to a biological measuring apparatus for measuring biological information of a user by using a worn type electronic device provided with a pulse wave measuring apparatus for measuring a pulse wave, a body temperature measuring apparatus for measuring a body temperature, a sweat measuring apparatus for measuring a sweating degree or the like, in particular, a body-mounted type exercise training support apparatus having a function of advising a user who exercises oneself.

### BACKGROUND ART

An electronic wrist watch is provided with a light emitting means such as a small lamp, a light emitting diode (LED), and an electro luminescence (EL) element in which a substance emits a light by energy obtained from an excitation source. Consequently, time information and so on can be read by irradiating a display device even in a dark place.

In recent years, there has been proposed an electronic wrist watch having a function of irradiating other than a display device or of emitting a light from a watch body. For such an electronic wrist watch, the watch body is provided with a light emitting means on the side face thereof for irradiating a periphery like a flashlight, or a light is emitted from a part of the watch body, thereby improving the fashionable property of an electronic wrist watch itself and a visibility from other persons.

In particular, for an electronic device configured in such a manner that a band that is connected to an electronic wrist watch body is provided with a light emitter, an area for emitting a light can be comparatively enlarged, thereby enabling a further light emitting production other than a light emission for the display device. In addition, the electronic wrist watch is outstanding and a visibility from a periphery can be improved, thereby improving a safety in the case in which the electronic wrist watch is worn at night. (As an example, see Patent document 1 (Japanese Patent Application Laid-Open Publication No. 11 (1999)-103912) and Patent document 2 (Japanese Registered Utility Model No. 3063339).)

The conventional art disclosed in Patent document 1 is provided with an EL element in a band of an electronic wrist watch. Patent document 1 describes that a light is emitted from the band by the EL element, thereby improving a fashionable property as jewelry or an accessory, and that the highly flexible EL element is used, thereby improving a durability of the light emitting means.

The conventional art will be described below with reference to the drawing. FIG. 23 is a figure redrawn in order to simplify the description of the conventional art disclosed in Patent document 1 without departing from the scope of the conventional art. A numeral 100 represents an electronic wrist watch body, a numeral 101 represents a band, a numeral 102 represents a casing, a numeral 103 represents a power source, a numeral 104 represents a switch means, a numeral 105 represents a battery, and a numeral 106 represents an EL element.

For the conventional art disclosed in Patent document 1, a light is emitted from the EL element 106 formed in the band 101 that is connected to the electronic wrist watch body 100. A power is supplied to the EL element 106 installed in the band 101 made of a transparent resin member or a semitransparent resin member from the battery 105 installed in the casing 102 connected to the band 101.

The power source 103 controls the power to be supplied from the battery 105 in such a manner that a method of a power supply to the EL element 106 is modified to a stationary supply, an intermittent supply, and a transient supply or the like, thereby generating a predetermined light emitting pattern. A vibration sensor (not shown) can be installed in the power source 103, and a user motion can be the trigger of a light emitting. The sensing of a vibration sensor (not shown) can be switched to an active one or an inactive one by operating the switch means 104 installed to the casing 102.

The conventional art disclosed in Patent document 2 is provided with an EL element in a dial and a band of an electronic wrist watch. The conventional art will be described below with reference to the drawing. FIG. 24 is a figure redrawn in order to simplify the description of the conventional art disclosed in Patent document 2 without departing from the scope of the conventional art.

A numeral 200 represents an electronic wrist watch body, a numeral 201 represents a dial, a numeral 202 represents a band, a numeral 203 represents a flexible panel, a numeral 204 represents a switch means, and numerals 291 to 298 represent EL elements. The EL elements 291 to 298, which can display a predetermined picture or figure (for instance, a dolphin), are disposed on the back face of the dial 201 and on the flexible panel 203. Lights are emitted from the EL elements 291 to 298 by current flowing.

The flexible panel 203 is disposed in the band 202. The band 202 is made of a member having a translucency in such a manner that lights emitted from the EL elements 291 to 298 are visible. Holes are formed in the dial 201 in such a manner that the EL elements 294 and 295 are visible.

The flexible panel 203 is disposed through an opening portion (not shown) of the electronic wrist watch body 200 and is electrically connected to a control section (not shown) disposed in the electronic wrist watch body 200. The control section controls a predetermined light emitting pattern of the EL elements 291 to 298 and enables a production effect such as a movement of a dolphin picture on the band 202 and from the band 202 to the dial 201.

The switch means 204 becomes a trigger for starting the light emission, or a vibration sensor or an angle sensor (not shown) that is a trigger for starting the light emission is activated or inactivated by the switch means 204.

Patent document 2 describes that a visibility in walking at night and an entertainment can be improved by such a configuration since lights are emitted from the dial and the band in an integrated manner.

It is said that the energy sources required for action of human beings are generally a protein, a sugar, and a lipid. The three sources are not always uniformly converted to energy but have an order of conversion corresponding to the conditions of action of human beings.

The protein of the three sources is converted last. As a protein, a muscle is decomposed to be energy after all of the sugar and the lipid is converted to energy. Consequently, in ordinary life and exercise conditions, the sugar and the lipid are used as energy sources.

The sugar and the lipid are differently used depending on exercise intensity. In the case in which exercise intensity is high, the sugar stored in a liver or a muscle is used as an energy source. To the contrary, in the case in which exercise intensity is low, the lipid is used as an energy source. Energy can be obtained by an oxygenic metabolism in which an energy source is decomposed by oxygen and an anoxic metabolism using no oxygen.

The energy that is obtained by an anoxic metabolism is utilized for exercise that requires an instantaneous force. However, the energy is small in such a manner that the energy source is exhausted by the full power exercise of approximately 40 seconds, and can be ignored in long-time exercise. Consequently, an energy consumption in long-time exercise is in almost proportion to an oxygen consumption amount.

Exercise intensity is an index that indicates intensity of exercise or a load to be applied to a body. And exercise intensity indicates a certain percentage of oxygen consumption during exercise in the case in which the maximum oxygen consumption amount is defined as 100%. As a value of the percentage is larger, energy consumption is larger, that is to say, a load to be applied to a body is larger.

A value of exercise intensity is not specified corresponding to exercise to be carried out or a type of exercise. Even in the case in which the identical exercise is carried out, a value of exercise intensity is different depending on each person. In order to know the precise value of exercise intensity, an oxygen consumption amount during exercise must be measured.

However, since a large-scale system and technical knowledge are required for the measurement, it is generally difficult to know the value of exercise intensity. Accordingly, in recent years, the value of exercise intensity has been obtained based on a heart rate in almost proportion to an oxygen consumption amount in many cases. That is to say, a load to be applied to a body can be known by a heart rate.

A heart rate is a value of a heart beat that is obtained in a certain period. In general, heart beats per one minute are adopted in many cases for easy understanding.

As described above, the lipid is used as an energy source during exercise having low exercise intensity, and the sugar is used as an energy source during exercise having high exercise intensity. Recently, in consideration of such conditions, many discussions have been performed to determine which of the sugar and the lipid is utilized as an energy source. In addition, many proposals have been made to apply such a thought to a development of body muscles, a diet, and a prevention of a life-style related disease or the like.

Moreover, in recent years, exercise intensity has been used for exercise training. For instance, it is reported that it is better to carry out exercise training in such a manner that exercise intensity is maintained to be the range of 70% to 80% in order to improve a stamina or a basic physical strength and to be the range of 40% to 60% in order to prevent a life-style related disease.

In consideration of such conditions, as one of methods for implementing a moderate amount of exercise training, it is known that a heart rate that is calculated from exercise intensity is used as an exercise training index, thereby adjusting an amount of exercise. That is to say, target exercise intensity should be set, and should be managed by using a target heart rate.

The target exercise intensity is the exercise intensity that is targeted when certain exercise training is started, and the corresponding heart rate is called a target heart rate. The target heart rate is calculated based on the following expression.
Target heart rate=(maximum heart rate−resting heart rate)×target exercise intensity+resting heart rate

It is difficult to measure the maximum heart rate, and the value of the maximum heart rate is generally reduced as age is increased. Therefore, it is known that the following expression can be substituted for the above expression.
Maximum heart rate=220−age

As described above, exercise intensity is not precisely specified for every exercise to be performed. However, it is known that there is a following relationship between the kind of exercise and exercise intensity. For instance, exercise intensity is 90% in the case of exercise of short-distance running at full speed, 60% in the case of exercise of jogging, and 40% in the case of exercise of walking.

In the case of a calculation of the above described target heart rate, a value of exercise intensity is input in consideration of the relationship between the kind of exercise and exercise intensity in many cases. For instance, it can be known that the target heart rate is in the range of 100 to 120 in the case in which a person who is forty years old is assumed to carryout exercise of jogging.

In the case in which a heart rate is measured, a finger is applied to a body side face of a wrist or a neck and senses a vibration of a skin corresponding to the pulsation of a heart. However, in the case in which it becomes popular that a target heart rate is set as an exercise training index of a person, a heart rate is desired to be measured even during or immediately after the exercise training. And a heart rate measuring instrument that can easily measure a heart rate even under such conditions has come to be required. In consideration of such conditions, many electronic heart rate measuring instruments have been proposed in recent years.

As such an electronic heart rate measuring instrument, an electrocardiogram (ECG) detection system is widely known in general. For instance, a pulse wave detector in a belt shape provided with an electrode for detecting a pulse wave is worn on a finger or a chest, and information is sent to a display portion disposed separately from the pulse wave detector by a wire or radio transmission for displaying the information.

The display portion is worn on an armor the like and displays a heart rate that is obtained based on the period of a pulse wave detected by the pulse wave detector. As a matter of course, a display portion can also be integrated with a pulse wave detector.

In addition to an electrode type provided with an electrode, the pulse wave detector involves an optical type that senses a change of a blood flow by light and a pressure type that senses a change of a blood pressure by a pressure.

As a matter of course, there have been proposed many types of apparatuses that can easily set a heart rate from user information such as age, a resting heart rate, a maximum heart rate, and target exercise intensity. In some proposals, the target heart rate having a range can be calculated, or the result that is obtained by comparing a heart rate being measured and user information can be informed of as an exercise training index by using characters, pictures, or sounds (as an example, see Patent document 3 (Japanese Patent Application Laid-Open Publication No. 9 (1997)-75491)).

The conventional art disclosed in Patent document 3 will be described below. FIG. 25 is a figure redrawn in order to simplify the description of the conventional art disclosed in Patent document 3 without departing from the scope of the conventional art. In FIG. 25, a numeral 299 represents a user, a numeral 300 represents an apparatus body, a numeral 301 represents a cable, a numeral 302 represents a sensor unit, a numeral 303 represents a wrist band, a numeral 308 represents a liquid crystal display device, and numerals 311a to 311g represent switches.

The apparatus body 300 is worn on an arm of the user 299 by using the wrist band 303. The sensor unit 302 is connected to the apparatus body 300 via the cable 301, and is worn on a finger of the user 299.

The apparatus body 300 carries out the signal processing of a pulse wave that is detected by the sensor unit 302 in order to obtain a heart rate, performs a calculation based on user information that is input from the switches 311a to 311g, and informs of information such as the obtained exercise training index and the heart rate. The information is informed of by a display of the liquid crystal display device 308 or an alarm sound made by a sounding means (not shown).

As described above, the conventional art disclosed in Patent document 3 can inform of an exercise training index by characters or sounds. By carrying out exercise in the state in which the apparatus body 300 that can measure a heart rate is worn, a user can know in real time whether or not the exercise is being carried out corresponding to a target heart rate that is the exercise training index, and can adjust an amount of exercise by utilizing the information.

Moreover, a user can also utilize the information in order to prevent so-called over training, in which the user is absorbed during exercise, thereby resulting in excessive exercise or departing from the normal physical condition.

[Patent document 1] Japanese Patent Application Laid-Open Publication No. 11 (1999)-103912 (page 4, FIG. 1)

[Patent document 2] Japanese Registered Utility Model No. 3063339 (page 2, FIG. 1)

[Patent document 3] Japanese Patent Application Laid-Open Publication No. 9 (1997)-75491 (pages 3 to 7, FIG. 3)

### DISCLOSURE OF THE INVENTION

### Problems to be Solved by the Invention

For the conventional arts disclosed in Patent documents 1 and 2, although a visibility can be improved by emitting a light from a band of an electronic wrist watch, the electronic wrist watch is disadvantageously inconvenient to use.

More specifically, for the conventional arts disclosed in Patent documents 1 and 2, a light emitter such as an EL element must be mounted into the band of the electronic wrist watch. Therefore, the structure of the band is more complicated than the structure of a band of a conventionally known electronic wrist watch, and a flexibility of the band is deteriorated, thereby degrading a fitting characteristic and a wearing characteristic to an arm, which is extremely important for an electronic wrist watch.

Moreover, there is a problem on a maintenance in which a length of a band cannot be adjusted (it is usually necessary to adjust a length of a band).

Furthermore, the manufacturing process of an electronic wristwatch is also complicated, thereby increasing a manufacturing cost.

Moreover, since a light emission area of a band itself is increased as compared with the case of emitting a light from only a display device such as a dial of an electronic wrist watch, a power consumption of the light emitter is increased. As a result, unfortunately, an operating time of an electronic wrist watch is shortened by consuming a power supply means such as a battery to be mounted to the electronic wrist watch.

The above problem can be solved by enlarging the power supply means. However, the solvent is not preferable since it is contrary to the recent requirement of the reduction in the size and the weight of an electronic wrist watch. In addition, the enlarged power supply means restricts a design of an electronic wrist watch.

Consequently, in practice, a proposal has not yet been submitted for implementing an electronic wrist watch that can all achieve an improvement of a visibility and a fashionable property due to a light emission of a band, a satisfactory wearing characteristic as an electronic wrist watch, and a sufficient operating time.

In addition to the above electronic wrist watch, there have been actively developed, for instance, worn type electronic devices having many functions such as a portable telephone of a type to be worn to an arm. In the case in which such an electronic device can inform by not only a sound such as a receiving sound but also the light emission from a band or an electronic device body as described above, a user can reliably find the information conveniently.

The present invention was made in order to solve the above problems of the conventional art. The objects of the present invention is to provide a worn type electronic device capable of simplifying the manufacturing and the maintenance of a band, reducing the restriction of a design, having a sufficient luminescent effect of a band, and reducing power consumption.

In recent years, many persons carry out exercise training at night and use a heart rate measuring instrument in a dark place. For the conventional art disclosed in Patent document 3, the liquid crystal display device 308 is provided with a back light. However, in the case in which a person carries out exercise in which the person's arms swing or the person's eye balls move up and down, the person cannot instantly find information such as an exercise training index unfortunately since the liquid crystal display device 308 is small.

In particular, since the liquid crystal display device 308 is disposed on the back side of a hand, an arm must be twisted to watch the liquid crystal display device 308. Therefore, for instance, an unnatural form is forced by watching the liquid crystal display device 308 while running, thereby disturbing a running form and normal exercise training.

Moreover, in use of the apparatus in the open air, information by sound cannot be heard by a noise of a car running on a public road and so on in many cases.

That is to say, the conventional art disclosed in Patent document 3 cannot sufficiently inform a user of information pertaining to exercise training disadvantageously. Such a problem is serious since the apparatus is used during exercise.

The present invention was made in order to solve the above problems of the conventional art. Another object of the present invention is to provide a biological measuring apparatus by which a training index can be easily recognized while a natural exercise form is maintained even in hard exercise or in an environment involving a noise.

### Means for Solving the Problems

The present invention was made in order to solve the above problems of the conventional art.

A worn type electronic device in accordance with the present invention is provided with a light emitting means for emitting a light from the worn type electronic device, and is characterized by comprising:

the light emitting means including a light emitting element for emitting a light having a specified wave length and a light guide means for guiding the light emitted from the light emitting element in a specified direction;

the light emitting element disposed in a body of the worn type electronic device, wherein a light emission of the light emitting element is controlled based on the control of the worn type electronic device; and

the light guide means including at least one of a body side light guide means disposed in the body of the worn type electronic device and a band side light guide means disposed in a band,

wherein the body side light guide means is disposed with a specified face made to face to the surface of the body of the worn type electronic device and the light emitted from the light emitting element is emitted from the surface of the body of the worn type electronic device via the body side light guide means; or

wherein the band side light guide means is disposed with a specified face made to face to the surface of the band and the light emitted from the light emitting element is emitted from the surface of the band via the band side light guide means.

The worn type electronic device in accordance with the present invention is characterized in that the light guide means is provided with a body side light guide means disposed in the body of the worn type electronic device and a band side light guide means disposed in a band, and the light emitted from the light emitting element is guided to the band side light guide means via the body side light guide means and is emitted from the surface of the band via the band side light guide means.

The worn type electronic device in accordance with the present invention is characterized in that the light guide means is provided with a body side light guide means disposed in the body of the worn type electronic device and a band side light guide means disposed in a band, the light emitted from the light emitting element is emitted from the surface of the body of the worn type electronic device via the body side light guide means, and the light emitted from the light emitting element is guided to the band side light guide means via the body side light guide means and is emitted from the surface of the band via the band side light guide means.

By such a configuration, the light emitted from the light emitting element disposed in the body of the worn type electronic device is guided by the light guide means and is emitted from the surface of the body of the worn type electronic device via the body side light guide means, or is emitted from the surface of the band via the band side light guide means. Moreover, the light emitted from the light emitting element can also be emitted from both the surface of the body of the worn type electronic device and the surface of the band.

Consequently, a light can be emitted from a part of the body of the worn type electronic device or the band. By this, in a light emission of the band for the worn type electronic device in accordance with the present invention, even in the case in which a light emitting means is not disposed in the band itself, the band looks as if it emitted a light from itself.

The light emission state of the band can be determined corresponding to a shape of the light guide means. Consequently, even in the case in which a light emission area of the band is increased, the power consumption of the worn type electronic device is not increased.

By such a configuration, the worn type electronic device such as an electronic wrist watch can all achieve an improvement of a visibility and a fashionable property due to a light emission of a band, a satisfactory wearing characteristic as an electronic wrist watch, and a sufficient operating time, without complicating the structure of the band.

There have been actively developed, for instance, worn type electronic devices having many functions such as a portable telephone of a type to be worn to an arm. Such electronic devices can inform by not only a sound such as a receiving sound but also the light emission from a band or an electronic device body as described above, a user can reliably find the information conveniently.

The worn type electronic device in accordance with the present invention is characterized in that the band side light guide means is disposed on the surface of the band or disposed in the band.

Since the band side light guide means is disposed on the surface of the band or disposed in the band as described above, the light that has been emitted from the light emitting element disposed in the body of the worn type electronic device can be guided via the surface or the inside of the band to be reliably emitted from the surface of the band. In addition, the structure of the band can be prevented from being complicated, and a visibility and a fashionable property due to a light emission of a band can be improved.

The worn type electronic device in accordance with the present invention is characterized in that a plurality of the light emitting elements is disposed in the body of the worn type electronic device and a plurality of the band side light guide means is disposed in the band corresponding to the light emitting elements.

Since a plurality of the light emitting elements is disposed in the body of the worn type electronic device and a plurality of the band side light guide means is disposed in the band corresponding to the light emitting elements as described above, more lights that have been emitted from the light emitting element can be transmitted to the band, and a light emission from the band can be more vivid. In addition, since a light emitting position can be freely selected, a light emitting position and a light emitting pattern can be freely produced.

The worn type electronic device in accordance with the present invention is characterized in that a width of the band side light guide means becomes narrower as the position on the band side light guide means becomes farther from the body of the worn type electronic device.

By such a configuration, in the case in which the worn type electronic device in accordance with the present invention is worn on an arm, the band in a six o'clock direction is disposed on a user side. Consequently, by enlarging a width of the band side light guide means section that is visible for a user, the band looks bright even in the case in which a light emitting element having the same luminance is used. Consequently, even in the case in which a drive power that is applied to the light emitting element is reduced, a light emitting state of the band is not so dark for a user, thereby reducing power consumption resulted from a drive of the light emitting element.

The worn type electronic device in accordance with the present invention is characterized by further comprising at least one shading index portion disposed on the surface of the band.

By such a configuration, the shading index portion disposed on the surface of the band can be, for instance, in a shape of a character, a symbol, or a figure. In the case in which a light enters the band side light guide means, only the periphery of the shading index portion disposed on the surface of the band is luminous, and a light to the shading index portion from a lower section is shaded. Consequently, a shape of the shading index portion can be conspicuous by a light emission, and the band can be luminous at one point in a predetermined shape or a predetermined pattern, thereby obtaining a high decorative effect.

The worn type electronic device in accordance with the present invention is characterized in that the surface of the band has a shading function and at least one index opening portion is formed on the surface of the band.

Since the surface of the band has a shading function and an index opening portion is formed on the surface of the band as described above, a light emitted from the band side light guide means is visible via the index opening portion.

Consequently, the index opening portion can be, for instance, in a shape of a character, a symbol, or a figure. In the case in which a light enters the band side light guide means, only the index opening portion disposed on the surface of the band is luminous, and a light to the periphery from an under section of the shading index portion is shaded. Consequently, a shape of the index opening portion disposed on the surface of the band can be conspicuous by a light emission, and the band can be luminous at one point in a predetermined shape or a predetermined pattern, thereby obtaining a high decorative effect.

The worn type electronic device in accordance with the present invention is characterized by further comprising at least one shading index portion disposed on the surface of the body side light guide means.

By such a configuration, the shading index portion disposed on the surface of the body side light guide means can be, for instance, in a shape of a character, a symbol, or a figure. In the case in which a light enters the body side light guide means, only the periphery of the shading index portion disposed on the surface of the body side light guide means is luminous, and a light to the shading index portion from a lower section is shaded. Consequently, a shape of the shading index portion can be conspicuous by a light emission, and the surface of the body side light guide means can be luminous at one point in a predetermined shape or a predetermined pattern, thereby obtaining a high decorative effect.

The worn type electronic device in accordance with the present invention is characterized in that the surface of the body side light guide means has a shading function and at least one index opening portion is formed on the surface of the body side light guide means.

Since the surface of the body side light guide means has a shading function and an index opening portion is formed on the surface of the body side light guide means as described above, a light emitted from the body side light guide means is visible via the index opening portion.

Consequently, the index opening portion can be, for instance, in a shape of a character, a symbol, or a figure. In the case in which a light enters the body side light guide means, only the index opening portion disposed on the surface of the body side light guide means is luminous, and a light to the periphery from an under section of the shading index portion is shaded. Consequently, a shape of the index opening portion disposed on the surface of the body side light guide means can be conspicuous by a light emission, and the band can be luminous at one point in a predetermined shape or a predetermined pattern, thereby obtaining a high decorative effect.

The worn type electronic device in accordance with the present invention is characterized by further comprising at least one filter on the surface of a light emission side of the light guide means, the filter for enabling only the light that has been emitted from the light emitting element and that has a specified wave length to pass through the filter.

By such a configuration, since only the light that has been emitted from the light emitting element and that has a specified wave length can pass through the filter, a user can be informed of information by the light having a specified wave length. In addition, a luminous color and a light emitting position can be freely selected by modifying a shape, a type, and a disposed position of the index portions. Consequently, a light emitting position and a light emitting pattern can be freely produced, thereby improving a visibility and a fashionable property due to a light emission.

The worn type electronic device in accordance with the present invention is characterized by further comprising at least one index portion on the surface of the filter, the index portion for emitting a light by the light that has been emitted from the light emitting element and passed through the filter and that has a specified wave length.

Since an index portion is formed on the surface of the filter and the index portion can emit a light by the light that has been emitted from the light emitting element and passed through the filter and that has a specified wave length as described above, a luminous color and a light emitting position can be freely selected by modifying a shape, a type, and a disposed position of the index portions. Consequently, a light emitting position and a light emitting pattern can be freely produced, thereby improving a visibility and a fashionable property due to a light emission.

The worn type electronic device in accordance with the present invention is characterized in that the light emitting element is composed of at least one light emitting element selected from an LED light emitting element, an ultraviolet light emitting element, and an electron beam emitting element.

By selecting a light emitting element as described above, a light can be emitted from a part of the body of the worn type electronic device or the band by a light having a specified wave length of various kinds, thereby improving a visibility and a fashionable property due to a light emission.

More specifically, in the case in which a light emitting element is an LED light emitting element, for instance, an LED light emitting element can be composed of the three color LEDs of a red (R) LED light emitting element, a green (G) LED light emitting element, and a blue (B) LED light emitting element, and each of lights of R, G, and B emitted from each of the above LEDs can pass through each of filters R, G, B, respectively. As a result, a light emitting position and a light emitting shape of R, G, and B can be selected, thereby improving a visibility and a fashionable property due to a light emission.

Moreover, In the case in which the light emitting element is an ultraviolet light emitting element (UV-LED) or an electron beam emitting element, a band-pass filter can be disposed in such a manner that the wave length of an ultraviolet light that is emitted from the UV-LED or an electron beam that is emitted from the electron beam emitting element is selected to make the light or beam pass through the band-pass filter. In addition, in the case in which a fluorescent substance that emits a light of a specified color by the ultraviolet light or electron beam is disposed, a light emitting position and a light emitting shape can be selected, thereby improving a visibility and a fashionable property due to a light emission.

The worn type electronic device in accordance with the present invention is characterized in that the band side light guide means is composed of an optical fiber.

By such a configuration, the light that has been emitted from the light emitting element disposed in the body of the worn type electronic device can be reliably guided via the optical fiber and can be reliably emitted from the surface of the band. In addition, the structure of the band can be prevented from being complicated, and a visibility and a fashionable property due to a light emission of a band can be improved.

The worn type electronic device in accordance with the present invention is characterized in that the band side light guide means is composed of a light guide member provided with diffraction gratings on the both faces thereof.

By such a configuration, the light that has been emitted from the light emitting element disposed in the body of the worn type electronic device can be reliably guided by the light guide member provided with the diffraction gratings on the both faces thereof, and can be diffracted at a gap of a slit of the diffraction grating to reliably emit the light from the surface of the band. In addition, the structure of the band can be prevented from being complicated, and a visibility and a fashionable property due to a light emission of a band can be improved.

The worn type electronic device in accordance with the present invention is characterized by further comprising:

at least one liquid crystal element disposed between the light emitting element and the band side light guide means, and

a plurality of index deflection members having deflection angles intersecting with each other at right angles on the surface of the band side light guide means,

wherein a plurality of the index deflection members is selected to diffuse a light by varying a voltage to be applied to the liquid crystal element.

By such a configuration, the light that has been emitted from the light emitting element disposed in the body of the worn type electronic device passes through the liquid crystal element and is guided to the band side light guide means.

By varying a voltage to be applied to the liquid crystal element, a light that has passed through the liquid crystal element can have a predetermined deflection angle. Consequently, a light can be emitted from the index deflection member that has a deflection angle corresponding to the above deflection angle and from the surface of the band.

Consequently, a light emitting position and a light emitting shape can be selected only by varying a voltage to be applied to the liquid crystal element, thereby improving a visibility and a fashionable property due to a light emission. In addition, the structure of the band can be prevented from being complicated, and a visibility and a fashionable property due to a light emission of a band can be improved.

The worn type electronic device in accordance with the present invention is characterized in that the light guide means is provided with a light storing means for storing a light guided by the light guide means.

The worn type electronic device in accordance with the present invention is characterized in that the light storing means is disposed adjacently to the light guide means.

The worn type electronic device in accordance with the present invention is characterized in that the light guide means provided with the light storing means is the band side light guide means.

As described above, in the case in which an absorption of a light and a light emission of the light storing member are utilized and the light emitting element is operated intermittently at a specified interval by a control means, a light emission of the light storing member continues even in a period in which a light emission of the light emitting element is not carried out, thereby maintaining a light emission of the body or the band of the worn type electronic device.

Consequently, a light can be continuously emitted from the body or the band of the worn type electronic device for a long time by extremely low power consumption.

A biological measuring apparatus provided with the worn type electronic device as defined in any one of the above descriptions for measuring biological information of a user is characterized in that a light emitted from the light emitting element is emitted from at least one of the surface of the body of the worn type electronic device and the surface of the band via at least one of the body side light guide means disposed in the body of the worn type electronic device and the band side light guide means disposed in the band in such a manner that biological information is informed of.

By such a configuration, the light emitted from the light emitting element disposed in the body of the worn type electronic device is guided by the light guide means and is emitted from the surface of the body of the worn type electronic device via the body side light guide means, or is emitted from the surface of the band via the band side light guide means. Moreover, the light emitted from the light emitting element can also be emitted from both the surface of the body of the worn type electronic device and the surface of the band. Consequently, biological information such as a heart rate and a body temperature can be reliably informed of.

The biological measuring apparatus in accordance with the present invention is characterized by further comprising:

a biological information value calculation means for detecting biological information of the user to obtain specified biological information values from the biological information;

a user information input means for inputting user information of the user;

a comparison operating means for comparing the user information and the biological information value; and

an informing means for informing of support information corresponding to the comparison results obtained by the comparison operating means,

wherein the informing means is provided with a first informing means for informing of support information in the case in which a light emitted from the light emitting element is emitted from at least one of the surface of the body of the worn type electronic device and the surface of the band via at least one of the body side light guide means disposed in the body of the worn type electronic device and the band side light guide means disposed in the band.

By such a configuration, biological information of a user is detected to obtain a biological information value by the biological information value calculation means, user information that has been input by a user in advance via the user information input means and the biological information value are compared by the comparison operating means, and support information is informed of by the informing means corresponding to the comparison results obtained by the comparison operating means.

By such a configuration, the light emitted from the light emitting element disposed in the body of the worn type electronic device is guided by the light guide means and is emitted from the surface of the body of the worn type electronic device via the body side light guide means, or is emitted from the surface of the band via the band side light guide means. Moreover, the light emitted from the light emitting element can also be emitted from both the surface of the body of the worn type electronic device and the surface of the band. Consequently, the informing means can reliably inform of support information such as a target heart rate, a training index indicating the relationship with any heart rate, and heart rate information.

The biological measuring apparatus in accordance with the present invention is characterized in that the informing means is further provided with a second informing means for informing of support information by using a character or a pictograph.

By such a configuration, the informing means is composed of the first informing means for informing of support information by using a light and the second informing means for informing of support information by using a character or a pictograph. Consequently, a light emission based on the first informing means enables a user to quickly know that there is information, and the second informing means enables a user to be informed of the detailed information.

Moreover, a light emission can be carried out based on any one of a light emitting position, a light emitting condition, and a light emitting pattern, or on the combination thereof, thereby improving a visibility and a variety in a representation.

As described above, since the roles of the first informing means and the second informing means are different from each other, information can be properly transmitted to a user.

The biological measuring apparatus in accordance with the present invention is characterized in that,

the biological measuring apparatus is a body-mounted type exercise training support apparatus having a function of advising a user who exercises oneself;

the biological information value calculation means is provided with a heart rate calculation means for detecting biological information of the user to calculate a heart rate from the biological information;

the comparison operating means is provided with a heart rate comparison operating means for comparing the user information and the heart rate; and

the informing means is provided with a heart rate informing means for informing of support information corresponding to the comparison results obtained by the heart rate comparison operating means.

By such a configuration, a pulse wave that is biological information of a user is detected to calculate a heart rate from the biological information. In addition, the informing means can reliably inform of a target heart rate to be calculated from a target exercise intensity that is user information that has been input in advance by a user, a training index indicating the relationship with any heart rate, and heart rate information. Consequently, informing means can be increased without modifying a limited space for a body-mounted type, and informing can be performed without complicating the structure of the body and the locking band.

The biological measuring apparatus in accordance with the present invention is characterized in that the first informing means of the informing means informs of support information prior to the time the second informing means informs of support information.

By such a configuration, a light emission based on the first informing means enables a user to quickly know that there is information.

The biological measuring apparatus in accordance with the present invention is characterized in that the biological measuring apparatus is at least one of a pulse wave measuring apparatus for measuring a pulse wave, a body temperature measuring apparatus for measuring a body temperature, and a sweat measuring apparatus for measuring a sweating degree, or a measuring apparatus in which said apparatuses are combined.

By such a configuration, the biological measuring apparatus in accordance with the present invention can be conveniently applied to the measurements of a pulse wave, a body temperature, and a sweating degree.

### EFFECT OF THE INVENTION

By the worn type electronic device in accordance with the present invention, the light emitted from the light emitting element disposed in the body of the worn type electronic device is guided by the light guide means and is emitted from the surface of the body of the worn type electronic device via the body side light guide means, or is emitted from the surface of the band via the band side light guide means. Moreover, the light emitted from the light emitting element can also be emitted from both the surface of the body of the worn type electronic device and the surface of the band.

Consequently, a light can be emitted from a part of the body of the worn type electronic device or the band. By this, in a light emission of the band for the worn type electronic device in accordance with the present invention, even in the case in which a light emitting means is not disposed in the band itself, the band looks as if it emitted a light from itself.

The light emission state of the band can be determined corresponding to a shape of the light guide means. Consequently, even in the case in which a light emission area of the band is increased, the power consumption of the worn type electronic device is not increased.

By such a configuration, the worn type electronic device such as an electronic wrist watch can all achieve an improvement of a visibility and a fashionable property due to a light emission of a band, a satisfactory wearing characteristic as an electronic wrist watch, and a sufficient operating time, without complicating the structure of the band.

There have been actively developed, for instance, worn type electronic devices having many functions such as a portable telephone of a type to be worn to an arm. Such electronic devices can inform by not only a sound such as a receiving sound but also the light emission from a band or an electronic device body as described above, a user can reliably find the information conveniently.

By the biological measuring apparatus provided with the worn type electronic device in accordance with the present invention, the light emitted from the light emitting element disposed in the body of the worn type electronic device is guided by the light guide means and is emitted from the surface of the body of the worn type electronic device via the body side light guide means, or is emitted from the surface of the band via the band side light guide means. Moreover, the light emitted from the light emitting element can also be emitted from both the surface of the body of the worn type electronic device and the surface of the band. Consequently, biological information such as a heart rate and a body temperature can be reliably informed of.

By the biological measuring apparatus provided with the worn type electronic device in accordance with the present invention, biological information of a user is detected to obtain a biological information value by the biological information value calculation means, user information that has been input by a user in advance via the user information input means and the biological information value are compared by the comparison operating means, and support information is informed of by the informing means corresponding to the comparison results obtained by the comparison operating means.

By such a configuration, the light emitted from the light emitting element disposed in the body of the worn type electronic device is guided by the light guide means and is emitted from the surface of the body of the worn type electronic device via the body side light guide means, or is emitted from the surface of the band via the band side light guide means. Moreover, the light emitted from the light emitting element can also be emitted from both the surface of the body of the worn type electronic device and the surface of the band. Consequently, the informing means can reliably inform of support information such as a target heart rate, a training index indicating the relationship with any heart rate, and heart rate information.

Moreover, the informing means is composed of the first informing means for informing of support information by using a light and the second informing means for informing of support information by using a character or a pictograph. Consequently, a light emission based on the first informing means enables a user to quickly know that there is information, and the second informing means enables a user to be informed of the detailed information.

Furthermore, a light emission can be carried out based on any one of a light emitting position, a light emitting condition, and a light emitting pattern, or on the combination thereof, thereby improving a visibility and a variety in a representation.

As described above, since the roles of the first informing means and the second informing means are different from each other, information can be properly transmitted to a user.

The biological measuring apparatus provided with the worn type electronic device in accordance with the present invention includes the first informing means and the second informing means. Support information is calculated from biological information that has been measured, and is informed of by the two informing means.

The first informing means carries out information with a light. A light emitting portion is disposed in an apparatus body or a locking band. In the case in which the light emitting portion is disposed in a locking band, a shape and a size of the light emitting portion in the band can be freely determined, and are not restricted by the size of the apparatus body, thereby enlarging the light emitting portion. Consequently, a user can obtain support information quickly even during exercise.

The informing means can also informs of only part of support information, such as information of only a heart rate that has been measured. Moreover, apparatus information can also be informed of. The apparatus information is information related to the biological measuring apparatus provided with the worn type electronic device in accordance with the present invention, such as information of a residual battery capacity.

In the case in which there is support information or apparatus information from the biological measuring apparatus provided with the worn type electronic device in accordance with the present invention, the first informing means informs of the information by utilizing a light in such a manner that a user can immediately notice the information. The second informing means displays more detailed information by using a character or a pictograph.

As described above, since the biological measuring apparatus provided with the worn type electronic device in accordance with the present invention is assumed to be used during exercise, the first informing means has a role for attracting user attention first, and the second informing means then informs of more detailed information. Such two-step information can prevent the oversight of support information.

### BRIEF DESCRIPTION OF THE DRAWINGS

FIG. 1 is a view illustrating an electronic wrist watch in accordance with the first embodiment of the present invention. FIG. 1(a) is an elevation view of the electronic wrist watch, and FIG. 1(b) is a side view of the electronic wrist watch, which is viewed from a six o'clock direction.

FIG. 2 is an elevation view illustrating an electronic wrist watch in accordance with the second embodiment of the present invention.

FIG. 3 is an elevation view illustrating an electronic wrist watch in accordance with the third embodiment of the present invention.

FIG. 4 is an elevation view illustrating an electronic wrist watch in accordance with the fourth embodiment of the present invention.

FIG. 5 is an elevation view illustrating an electronic wrist watch in accordance with the fifth embodiment of the present invention.

FIG. 6 is a view illustrating an electronic wrist watch in accordance with the sixth embodiment of the present invention. FIG. 6(a) is a schematic side view showing the internal configuration of the electronic wrist watch, which is viewed from the side in a three o'clock direction. FIG. 6(b) is a side view of the electronic wrist watch, which is viewed from a band 4 in a six o'clock direction.

FIG. 7 is a view illustrating an electronic wrist watch in accordance with the seventh embodiment of the present invention. FIG. 7(a) is an elevation view of the electronic wrist watch, and FIG. 7(b) is a schematic side view showing the internal configuration of the electronic wrist watch, which is viewed from the side in a three o'clock direction.

FIG. 8 is a view illustrating an electronic wrist watch in accordance with the eighth embodiment of the present invention. FIG. 8(a) is an elevation view of the electronic wrist watch, and FIG. 8(b) is a schematic side view showing the internal configuration of the electronic wrist watch, which is viewed from the side in a three o'clock direction.

FIG. 9 is an elevation view illustrating an electronic wrist watch in accordance with a ninth embodiment of the present invention.

FIG. 10 is an elevation view illustrating an electronic wrist watch in accordance with a tenth embodiment of the present invention.

FIG. 11 is a schematic side view showing the internal configuration of a body-mounted type exercise training support apparatus in accordance with the eleventh embodiment of the present invention, which is viewed from the side in a three o'clock direction.

FIG. 12 is a block diagram for illustrating the electric circuit of a body-mounted type exercise training support apparatus in accordance with the present invention.

FIG. 13 is an elevation view illustrating an embodiment in which information is displayed on a display device 2 that is a second informing means.

FIG. 14 is an elevation view illustrating an electronic wrist watch in accordance with the twelfth embodiment of the present invention.

FIG. 15 is a schematic side view showing the internal configuration of the electronic wrist watch shown in FIG. 14, which is viewed from the side in a three o'clock direction.

FIG. 16 is a schematic side view illustrating an electronic wrist watch in accordance with the thirteenth embodiment of the present invention and showing the internal configuration of the electronic wrist watch, which is viewed from the side in a three o'clock direction.

FIG. 17 is a schematic side view illustrating an electronic wrist watch in accordance with the fourteenth embodiment of the present invention and showing the internal configuration of the electronic wrist watch, which is viewed from the side in a three o'clock direction.

FIG. 18 is a schematic side view illustrating an electronic wrist watch in accordance with the fifteenth embodiment of the present invention and showing the internal configuration of the electronic wrist watch, which is viewed from the side in a three o'clock direction.

FIG. 19 is a schematic side view illustrating an electronic wrist watch in accordance with the sixteenth embodiment of the present invention and showing the internal configuration of the electronic wrist watch, which is viewed from the side in a three o'clock direction.

FIG. 20 is an elevation view of the electronic wrist watch shown in FIG. 19.

FIG. 21 is a schematic side view illustrating an electronic wrist watch in accordance with the seventeenth embodiment of the present invention and showing the internal configuration of the electronic wrist watch, which is viewed from the side in a three o'clock direction.

FIG. 22 is a circuit block diagram of the electronic wrist watch shown in FIG. 21.

FIG. 23 is an elevation view showing a conventional electronic wrist watch.

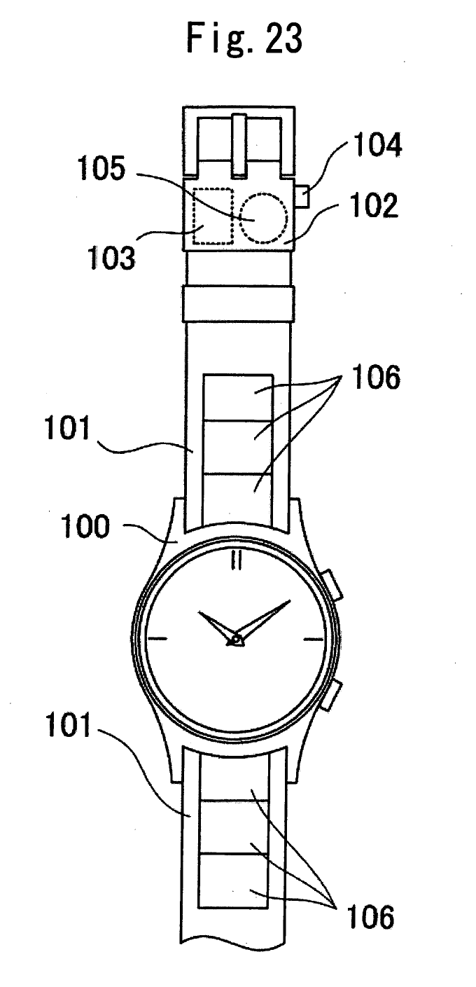

FIG. 24 is an elevation view showing a conventional electronic wrist watch.

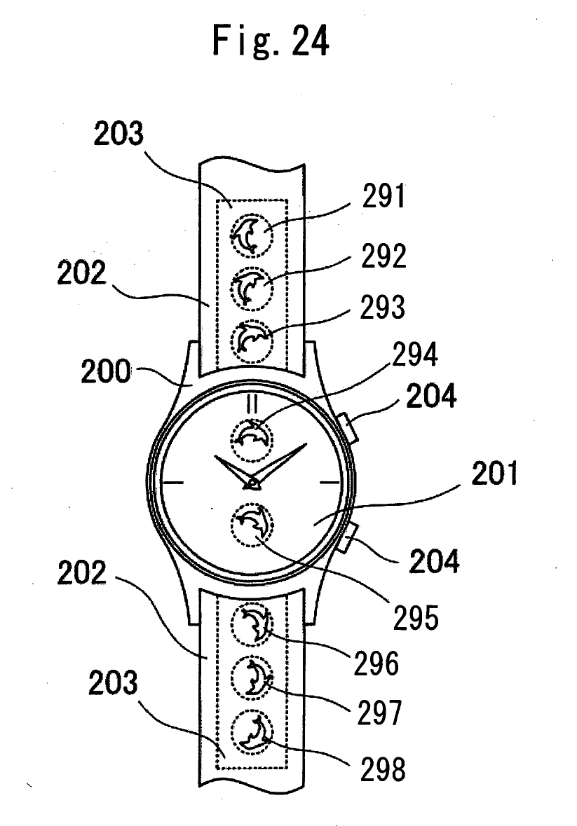

FIG. 25 is an elevation view showing a conventional body-mounted type exercise training support apparatus.

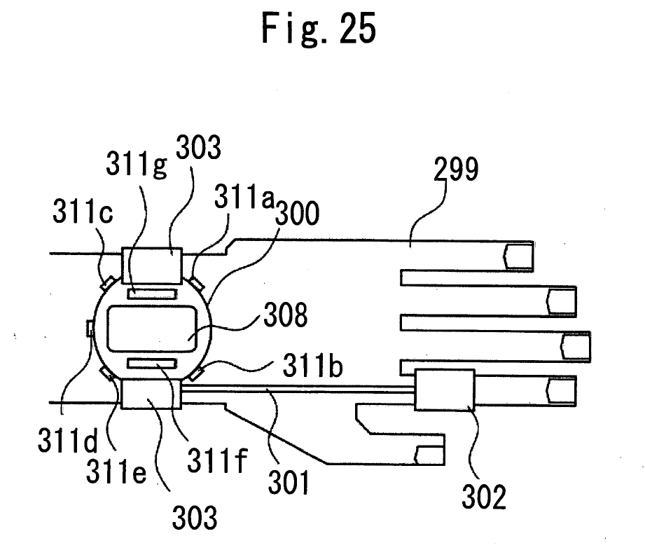

### EXPLANATIONS OF LETTERS OR NUMERALS

1: body case
2: display device
3: switch means
4: band
5: opening portion
6: light emitting element
6a, 6b, 6c: light emitting elements
7: light guide means
7a, 7b: light guide means
8: shading member
11: windbreaking glass
24: pulse wave detector
26: light emission driving circuit
27: display driving circuit
30: shading member
31: opening portion
32: protruding portion
34: light emitting element
35: red (R) filter
36: green (G) filter
37: blue (B) filter
38: red (R) index portion
39: green (G) index portion
40: blue (B) index portion
41: segment
51: dot matrix display area
52: segment display area
53: target heart rate display
54: residual battery capacity display
55: heart rate display
56: icon
57: icon
61: ultraviolet light emitting element
62 to 64: band-pass filters
65 to 67: index portions
68: ultraviolet filter
69: optical fiber
70a, 70b, 70c: opening portions
71: first light guide means
72: second light guide means
80: core
81: clad
82, 83: diffraction gratings
84: light guide member
85: incident prism
86: liquid crystal element
87, 88: index deflection member
89: LED for signal
90: photo detector
91: FPC (flexible printed circuit board)
92: LED for display
93: battery for display
94: battery for watch
95, 96: optical path modification means
99: user
100: electronic wrist watch body
101: band
102: casing
103: power source
104: switch means
105: battery
106: EL element
200: electronic wrist watch body
201: dial
202: band
203: flexible panel
204: switch means
291 to 298: EL elements
299: user
300: apparatus body
301: cable
302: sensor unit
303: wrist band
308: liquid crystal display device
X: virtual line

### BEST MODE FOR CARRYING OUT THE INVENTION

An embodiment (example) of the present invention will be described below in detail with reference to the drawings.

### Embodiment 1

### First Embodiment

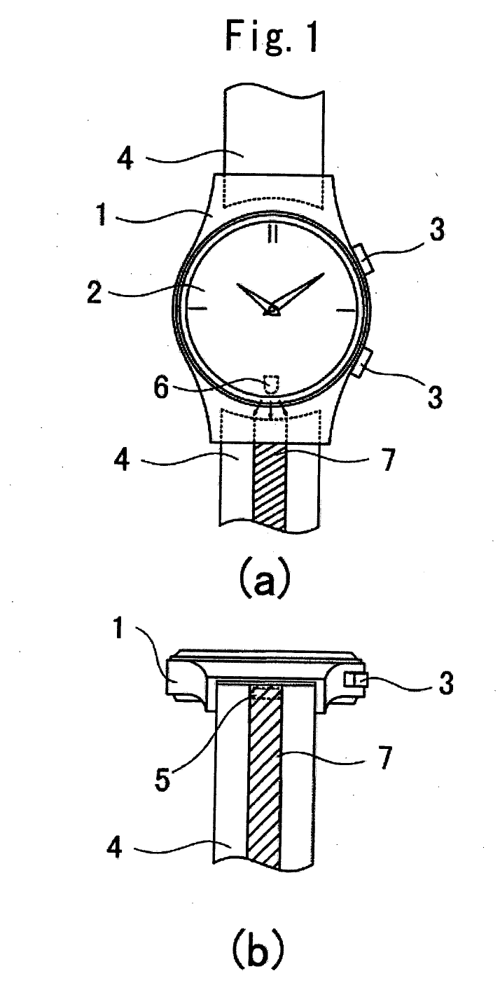

### FIG. 1

With reference to the drawings, the following describes in detail a best mode in the case in which the present invention is applied to an electronic wrist watch as a worn type electronic device.

FIG. 1 is a view illustrating an electronic wrist watch in accordance with the first embodiment of the present invention. FIG. 1(a) is an elevation view of the electronic wrist watch, and FIG. 1(b) is a side view of the electronic wrist watch, which is viewed from a six o'clock direction.

In these figures, a numeral 1 represents a body case, a numeral 2 represents a display device, a numeral 3 represents a switch means, a numeral 4 represents a band, a numeral 5 represents an opening portion, a numeral 6 represents a light emitting element, and a numeral 7 represents a light guide means. In FIG. 1, lights that are emitted from the light emitting element 6 are shown by arrows.

The light emitting element 6 is disposed inside the body case 1, and the light guide means 7 is disposed in the band 4. Consequently, the light that has been emitted from the light emitting element 6 passes through the opening portion 5 formed in a six o'clock direction of the body case 1 and enters the light guide means 7 disposed in the band 4. The light guide means 7 can guide the light that has been emitted from the light emitting element 6 in a specified direction.

The specified direction means a direction in which a light should be emitted, for instance, the surface direction of the body case 1 or the band 4. The light guide means 7 is disposed in such a manner that a specified face is oriented to the above specified direction. The specified face is a plane or an end face of the light guide means 7 from which a light is emitted.

By such a configuration, an incident light is guided to a direction of the specified face and visible for a user. Even in the case in which a light emitting element is not disposed on the band 4 itself, a user can see the band 4 as if the band were emitting a light from itself.

The opening portion 5 is formed in the body case 1 in such a manner that the light emitting element 6 formed in the body case 1 and the light guide means 7 formed in the band 4 are disposed facing to each other without a shielding substance therebetween in the case in which the end face of the band 4 is fitted into the body case 1. By such a configuration, the light that has been emitted from the light emitting element 6 reaches the light guide means 7 without being shielded by the body case 1, and the light guide means 7 can emit the light in the specified direction. The band 4 and the body case 1 can be connected to each other using a band fitting structure having become common property for a watch. Consequently, the detailed descriptions of the structure are omitted.

The display device 2 is illustrated using an example of a dial of a so-called analog display type, which informs of time by watch hands. However, the display device is not restricted to the analog display type as a matter of course, and a so-called digital display type dial can also be adopted for informing of time by numerals and pictographs.

The body case 1 or the band 4 can be made of publicly known metal raw materials or resin raw materials. The embodiment shown in FIG. 1 illustrates an example in which the band is composed of flexible resin raw material or leather raw material. However, the band is not restricted to such configurations as a matter of course, and the band can also be in a publicly known structure in which a plurality of segments are linked at rotating parts or be a structure like a bracelet. The band configuration using a plurality of the segments is described later.

While the light emitting element 6 is not restricted in particular, a light emitting diode (LED) can be used. Although an electro luminescence (EL) element and an electric light bulb can also be used, it is preferable to use a light emitter that requires less power consumption.

The embodiment in accordance with the present invention does not restrict a luminescent color in particular. However, in consideration of a visibility and a fashionable property (representation) due to a light emission, multiple colors are preferably used as a luminescent color. For instance, it is preferable to use a color LED that can emit white, blue, green, yellow, orange, red and so on by controlling a driving electric current to be applied to the LED, and a plurality of the color LEDs can also be combined.

In the case in which a light emitting element is an LED light emitting element, for instance, an LED light emitting element can be composed of the three color LEDs of a red (R) LED light emitting element, a green (G) LED light emitting element, and a blue (B) LED light emitting element, and each of lights of R, G, and B emitted from each of the above LEDs can pass through each of filters R, G, B, respectively. As a result, a light emitting position and a light emitting shape of R, G, and B can be selected, thereby improving a visibility and a fashionable property due to a light emission.

As a matter of course, a light to be emitted is not restricted to a visible light, and an ultraviolet light or the like can also be used. For instance, an ultraviolet light having a wave length of 365 nm can be used. In the case in which an aqueous solution or a raw material that contains vitamin B (riboflavin) is sealed in a light guide means 7 described later, and an invisible light flux in an ultraviolet region is irradiated, then a greenish yellow colored fluorescent light is emitted from the light guide means 7.

More specifically, as described later, the light emitting element can be an ultraviolet light emitting element (UV-LED) or an electron beam emitting element. In this case, a band-pass filter can be disposed in such a manner that the wave length of an ultraviolet light that is emitted from the UV-LED or an electron beam that is emitted from the electron beam emitting element is selected to make the light or beam pass through the band-pass filter. In addition, in the case in which a fluorescent substance that emits a light of a specified color by being irradiated the ultraviolet light or electron beam is disposed, a light emitting position and a light emitting shape can be selected, thereby improving a visibility and a fashionable property due to a light emission.

The light guide means 7 can guide a light in a specified direction by utilizing a reflection caused by the difference in refractive indexes between air and the material forming the light guide means. A publicly known member can be used as the light guide means 7. Although it is not restricted in particular, the light guide means 7 can be composed of a transparent plate made of an acrylic resin, and a fine pattern is printed or patterned in order to diffuse lights uniformly to a specified face.

As a matter of course, a light guide means that has been adopted for a back light of a publicly known liquid crystal display device or the like can also be used, and an optical fiber can also be used.

A publicly known light scattering member can be bonded to a part or the entire of the surface of the light guide means 7. Moreover, a half mirror processing layer, a reflecting member, a colored layer, or a plating processing layer can also be formed.

It is important that the light that has been emitted from the light emitting element 6 is guided in a specified direction. It is more preferable in consideration of an improvement of a visibility and a fashionable property to uniform a luminous lightness of the light guide means 7 and to improve decorativeness during a non-emission state.

A publicly known light storing member can be disposed adjacently to the light guide means 7 in the band 4.

A control means such as a CPU 21 described later in the electronic wrist watch operates intermittently the light emitting element 6 at a specified interval. And a light emission of the light storing member continues even in a period in which a light emission of the light emitting element 6 is not carried out, utilizing the light absorption and the light emission of the light storing member. Thereby the light emission of the band 4 is maintained. Consequently, a light can be continuously emitted from the band 4 for a long time by extremely low power consumption.

The switch means 3 is electrically connected to a control means (not shown) of the electronic wrist watch, and is operated by a user in order to control a light emission of the light emitting element 6. For instance, the switch means 3 enables a continuous lighting, a blink, an intermittent lighting, turning out of the light, and a modification of a luminous color or the like.

Moreover, a light can be emitted from the light emitting element 6 by using a signal that is input from a sensing means (not shown) as a trigger. The switch means 3 can activate or inactivate the sensing means.

As the sensing means, there are mentioned, for instance, a vibration sensor for outputting a signal by a detection of a vibration, an inclination sensor for outputting a signal by a detection of an inclination, an acceleration sensor for outputting a signal by a detection of an acceleration, a temperature sensor, an atmospheric pressure sensor, and a sensor for outputting a signal by a detection of receiving of a radio wave in an information apparatus provided with a communication means.

### Second Embodiment

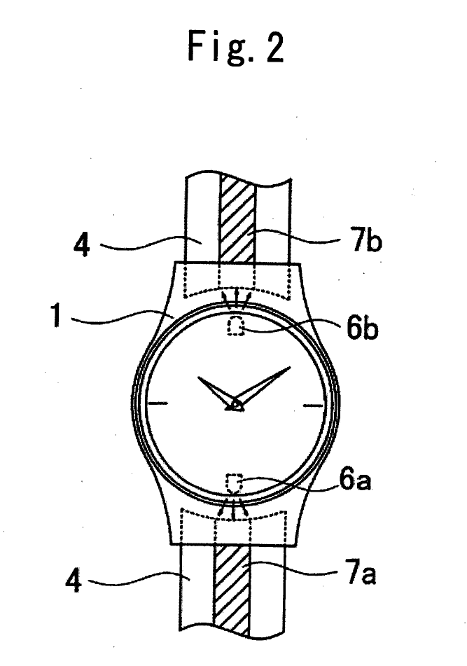

### FIG. 2

FIG. 2 is an elevation view illustrating an electronic wrist watch in accordance with the second embodiment of the present invention. A light emitting element 6a and a light guide means 7a are disposed in a six o'clock direction of the electronic wrist watch, and a light emitting element 6b and a light guide means 7b are disposed in a twelve o'clock direction. The light emitting elements 6a and 6b emit a light in a direction of the band 4 on the side in which each light emitting element is disposed, and the light guide means 7a and 7b receive the light. Each of the light guide means guides the received light to make the light visible for a user.

As a matter of course, the light emitting elements 6a and 6b can be blinked simultaneously or alternately each other, and can emit lights with different colors, respectively.

By such a configuration, a light emitting area of the band 4 can be enlarged, thereby further improving a visibility not only for a user but also from a periphery.

### Third Embodiment

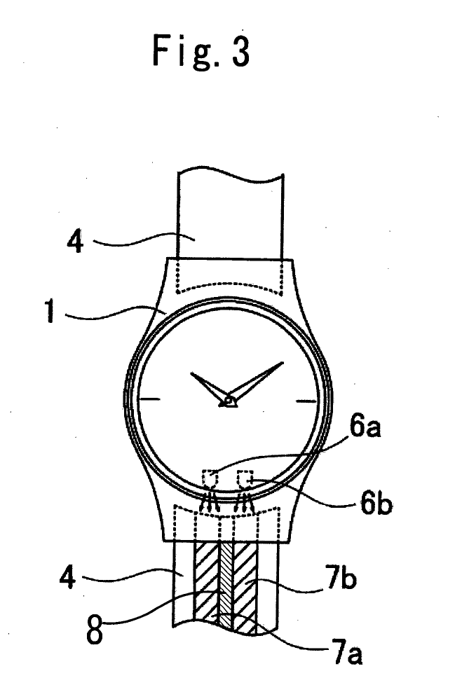

### FIG. 3

FIG. 3 is an elevation view illustrating an electronic wrist watch in accordance with the third embodiment of the present invention. The light emitting elements 6a and 6b and the light guide means 7a and 7b are disposed in a six o'clock direction of the electronic wrist watch.

A shading member 8 is disposed between the two light guide means 7a and 7b. The shading member 8 can prevent the light emission and the light guiding of the light guide means 7a and those of the light guide means 7b from being interfered with each other.

By such a configuration, each of the light emitting elements enables a light emission of the light guide means disposed separately at the specified position of the band 4. Consequently, in the case in which a light emission of the light emitting element is utilized as an informing means, information to be informed of can be modified corresponding to a light emitting state of the light guide means 7a and 7b, and information to be informed of can have specified meanings corresponding to a combination of the light guide means 7a and 7b.

For instance, a red light and a blue light can be emitted from the light guide means 7a and 7b, respectively. Moreover, only one light guide means, for example the light guide means 7b, can be blinked at every just o'clock. As described above, different color light emissions from the light guide means 7a and 7b enable to inform the information having more a satisfactory visibility and a variety in a representation to be informed of and a decorative effect to be obtained.

While the embodiment shown in FIG. 3 illustrates the case in which the above structure is formed in a six o'clock direction of the body case 1, the structure can also be formed in a twelve o'clock direction of the body case 1, and in both six and twelve o'clock directions as a matter of course.

### Fourth Embodiment

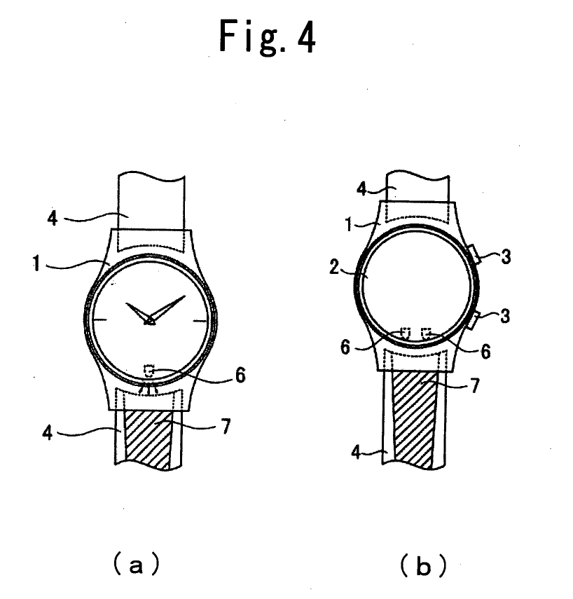

### FIG. 4

FIGS. 4(a) and 4(b) are elevation views illustrating an electronic wrist watch in accordance with the fourth embodiment of the present invention. A light emitting element 6 and a light guide means 7 are disposed in a six o'clock direction of the electronic wrist watch. The shape of the light guide means 7 is different from that of the first embodiment. In the case in which the width of the light guide means 7 is defined as the width along the transverse direction of the band 4, the width of the light guide means becomes narrower as the position on the light guide means 7 becomes farther from the body case 1.

In the case in which the electronic wrist watch that is a worn type electronic device in accordance with the present invention is worn on an arm, the side of the band 4 in the six o'clock direction is disposed on a user side. Consequently, by enlarging a width of the light guide means section that is easily noticed by a user, the band 4 looks brighter even in the case in which the luminescence intensity of the light emitting element 6 is not improved.

Consequently, even in the case in which a drive power that is applied to the light emitting element 6 is reduced, a light emitting state of the band 4 is not so dark for a user, thereby the power consumption during driving the light emitting element 6 can be reduced.

While the embodiment shown in FIG. 4(b) illustrates the case in which two light emitting elements 6 are disposed, the present invention is not restricted to the case. The number of the light emitting elements 6 can be selected without restriction in consideration of the luminance and the power consumption of the LED that configures the light emitting element 6. As a matter of course, each of the two light emitting elements can be blinked alternately, and can emit lights with different colors.

While the embodiment shown in FIG. 4 illustrates the case in which the light guide means 7 is disposed in the six o'clock direction of the body case 1, the light guide means 7 in such a shape can also be disposed in the twelve o'clock direction of the body case 1 as a matter of course. By such a configuration, since a light is emitted from the band 4 disposed in a twelve o'clock direction, the luminescence can become easy to be seen not only for a user but also for third persons, thereby improving a visibility and a variety in a representation for third persons. For instance, if a user enables the light emission during a going out at night, a visibility can be improved for drivers of other means of transport. Thereby contingencies such as traffic accidents can be prevented. As a matter of course, the light guide means 7 can be disposed in both six and twelve o'clock directions.

### Fifth Embodiment

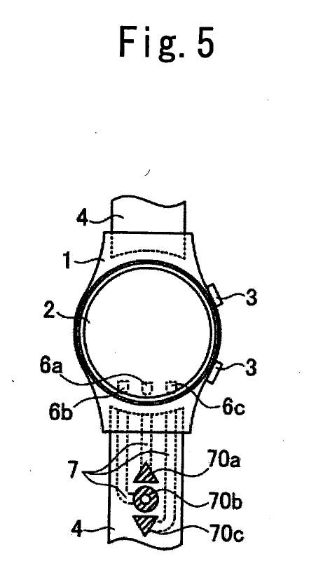

### FIG. 5

FIG. 5 is an elevation view illustrating an electronic wrist watch in accordance with the fifth embodiment of the present invention.

As shown in FIG. 5, light emitting elements 6a, 6b, and 6c, corresponding plural light guide means 7, and opening portions 70a, 70b, and 70c are disposed in a six o'clock direction of the electronic wrist watch in accordance with the present invention.

In FIG. 5, the light emitting elements 6a, 6b, and 6c, and the opening portions 70a, 70b, and 70c are disposed on the parts of the surfaces of each light guide means 7 in the six o'clock direction of the electronic wrist watch that is a worn type electronic device in accordance with the present invention. The light emissions of each light guide means 7 are visible from the opening portions 70a, 70b, and 70c. Shading members (not shown) are disposed between the light guide means 7, and can prevent the light emissions and the light guides of one light guide means and those of other light guide means from being interfered with each other.

The opening portions 70a, 70b, and 70c can be in a shape of a character, a symbol, or a figure. By such a configuration, a light emission can be more conspicuous.

Moreover, the light emissions can also be controlled independently corresponding to each of the light emitting elements 6a, 6b, and 6c. For instance, in the case in which a worn type electronic device in accordance with the present invention is utilized as a biological measuring apparatus of an electronic wrist watch type. And the state of certain training is informed of by using certain measured biological information. The light emitting element 6a is blinked for a predetermined period in the state of overtraining against a target. The light emitting element 6c is blinked for a predetermined period in the state of under-training against a target. And the light emitting element 6b is blinked for a predetermined period in the state of target training. As a matter of course, a modification of a luminous color can further call an attention.

Such a configuration enables information having a more satisfactory visibility and a variety in a representation to be informed. Moreover, the opening portions 70a, 70b, and 70c can be sealed with a transparent resin or a semitransparent resin, thereby improving a quality and decorativeness in a period in which a light emission is not carried out.

While the embodiment illustrated in FIG. 5 shows the case in which the structure described above is formed in the six o'clock direction of the body case 1. The structure can also be formed in the twelve o'clock direction, or in both the six o'clock direction and twelve o'clock direction of the body case 1 as a matter of course.

### Sixth Embodiment

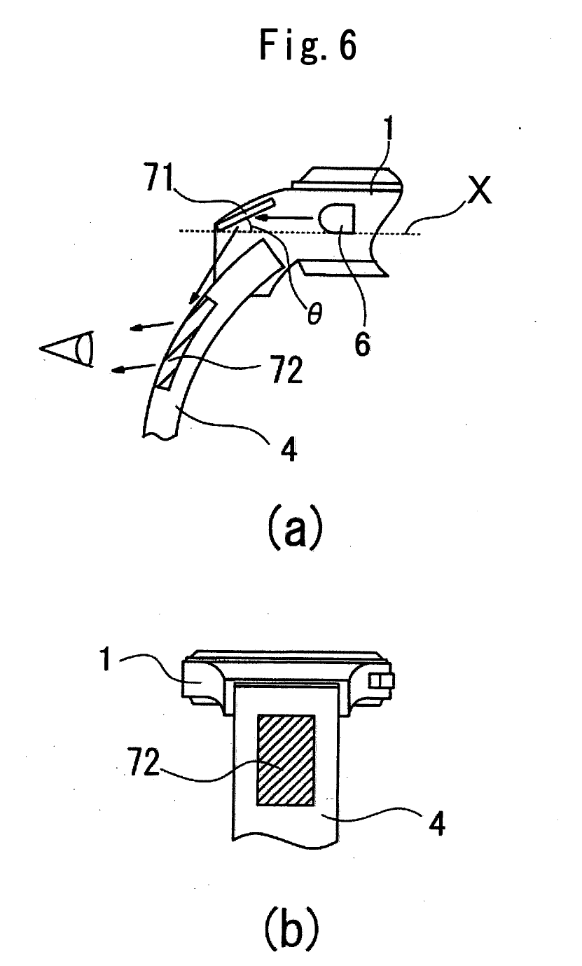

### FIG. 6

FIG. 6 is a view illustrating an electronic wrist watch in accordance with the sixth embodiment of the present invention. FIG. 6(a) is a schematic side view showing the internal configuration of the electronic wrist watch, which is viewed from the side in a three o'clock direction. FIG. 6(b) is a side view of the electronic wrist watch, which is viewed from the band 4 in a six o'clock direction.

The embodiment shown in FIG. 6 illustrates the case in which two light guide means are used for one light emitting element. A numeral 71 represents the first light guide means, a numeral 72 represents the second light guide means, and the letter X represents a virtual line.

As shown in FIG. 6(a), the first light guide means 71 composed of a reflecting member is disposed around the end portion in the six o'clock direction in the body case 1. And the first light guide means 71 guides the light that has been emitted from the light emitting element 6 to the second light guide means 72 disposed on the band 4.

Moreover, the first light guide means 71 has a specified angle θ against the virtual line X lined parallel to the display device 2 (not shown). And the first light guide means 71 reflects the light that has been emitted from the light emitting element 6 to the direction of the surface of the band 4 disposed in the six o'clock direction of the body case 1 without scattering or diffusion.

The second light guide means 72 is composed of a reflecting member and reflects the incident light to a specified direction such as the direction of the surface of the band 4 without scattering or diffusion. In FIG. 6(a), arrows indicate the light that has been emitted from the light emitting element 6.

While FIG. 6(a) shows an embodiment in which the arrows indicate that the guided light passes through outside of the body case 1 from the first light guide means 71 to the second light guide means 72, the present invention is not restricted to the embodiment.

A light that is guided by the first light guide means 71 can also reach the second light guide means 72 through inside of the band 4.

In such a case, the second light guide means 72 is not a reflecting member but a light guide means for guiding a light in a specified direction as described above. By such a configuration, the light of the light emitting element 6 can be emitted from the specified face of the second light guide means 72.

As shown in FIG. 6(b), the second light guide means 72 can be disposed on the band 4 apart from the body case 1. Consequently, a light can be emitted from the band 4 without restriction pertaining to a design.

The light emitting element 6 can also be composed of a light emitting element that emits a light having a high directivity. In such a case, the light that has been emitted from the light emitting element 6 can be guided to the face of a user via the light guiding from the first light guide means 71 to the second light guide means 72 without scattering or diffusion. By such a configuration, only a user can check the light of a band without illuminating the periphery.

For instance, even in the place in which an inconvenience occurs if the periphery is illuminated, such as a movie theater and a cabin of an air plane at lights-out, a light of the band is visible for only a user without illuminating the periphery.

The case in which the sensing means described above is mounted to the electronic wrist watch that is the worn type electronic device in accordance with the present invention and a light is emitted from the band 4 based on the information output from the sensing means is assumable. And the light is unexpectedly emitted in some cases. Even in such a condition, the light that has been emitted from the light emitting element 6 can be guided to the face of a user via the light guiding from the first light guide means 71 to the second light guide means 72 without scattering or diffusion. By such a configuration, a light emission is visible for a user without being aware of the light emission for a peripheral third person.

As described above, since the light guide means is composed of the first light guide means 71 and the second light guide means 72, a light can be emitted from the band 4 even in the case in which the light guide means is not extended to the section in the vicinity of the light emitting element 6 disposed in the body case 1.

### Seventh Embodiment

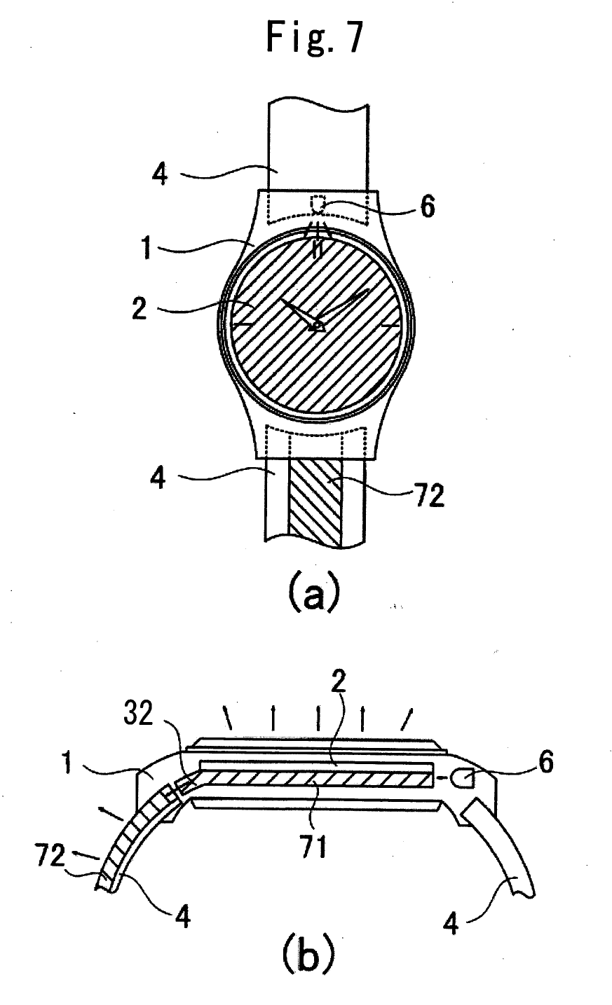

### FIG. 7

FIG. 7 is a view illustrating an electronic wrist watch in accordance with the seventh embodiment of the present invention. FIG. 7(a) is an elevation view of the electronic wrist watch, and FIG. 7(b) is a schematic side view showing the internal configuration of the electronic wrist watch, which is viewed from the side in a three o'clock direction.

In FIG. 7, the numeral 32 represents a protruding portion that is formed at an end of a first light guide means 71, and the light that has been emitted from the light emitting element 6 is indicated by arrows.

In the embodiment shown in FIG. 7, the light emitting element 6 is disposed in the twelve o'clock direction of the body case 1. The display device 2 is, for instance, a dial of an analog display type, which informs of time by watch hands, and is made of a translucent member. The first light guide means 71 is disposed on the backside of the display device 2.

The first light guide means 71 can guide a light in a direction of the display device 2, and is provided with the protruding portion 32 at the end in a six o'clock direction. The protruding portion 32 has a light guiding function for guiding the light that has entered the first light guide means 71 toward the second light guide means 72 disposed on the band 4.

More specifically, the light that has been emitted from the light emitting element 6 is guided to the front face of the electronic wrist watch in accordance with the present invention through the display device 2 by the first light guide means 71. Consequently, the display device 2 looks as if it emitted a light from itself. The light is guided to the second light guide means 72 by the protruding portion 32, and is emitted from the surface of the band 4.

By such a configuration, the light that is emitted from the light emitting element 6 not only makes the band 4 luminous but also illuminates the display device 2. Consequently, an illumination means that is required for recognizing the time on the display device 2 is not required, thereby miniaturizing the electronic wrist watch in accordance with the present invention. As a matter of course, since a light is also emitted from the display device 2, a further decorative effect can be obtained.

The positional relation between the protruding portion 32 and the light emitting element 6 is not restricted to the embodiment shown in FIG. 7. The light emitting element 6 can also be disposed in the three o'clock direction of the body case 1 in such a manner that a light is irradiated in the center direction of the display device 2, and the protruding portion 32 can be disposed in both six and twelve o'clock directions in such a manner that a light is guided to the second light guide means 72 formed in the bands 4 disposed in the both directions. In such a case, the light emitting element 6 can also be disposed in a nine o'clock direction or a three o'clock direction. That is to say, the position of the light emitting element 6 is not restricted in particular, and can be modified as needed.

Moreover, the protruding portion 32 can be formed separately from the first light guide means 71.

Furthermore, while FIG. 7(b) shows the embodiment in which the end face of the protruding portion 32 and the end face of the second light guide means 72 are disposed in parallel slightly apart from each other, the two end faces can come into contact with each other.

### Eighth Embodiment

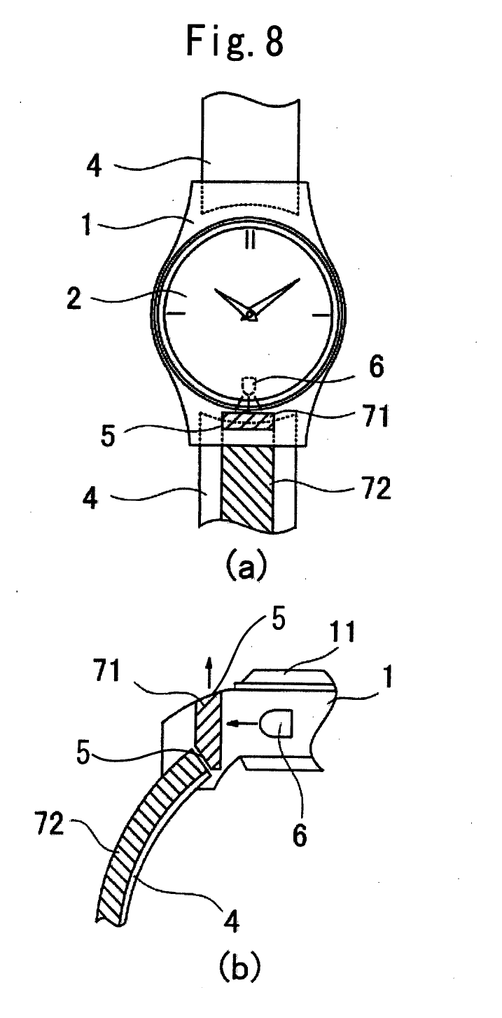

### FIG. 8

FIG. 8 is a view illustrating an electronic wrist watch in accordance with an eighth embodiment of the present invention. FIG. 8(a) is an elevation view of the electronic wrist watch, and FIG. 8(b) is a schematic side view showing the internal configuration of the electronic wrist watch, which is viewed from the side in a three o'clock direction.

In FIG. 8, a numeral 5 represents an opening portion that is formed in a body case 1, and a light that has been emitted from a light emitting element 6 is indicated by an arrow. A numeral 11 represents a windbreaking glass for protecting a display device 2.

In the embodiment shown in FIG. 8, the opening portion 5 is formed in a direction of the windbreaking glass 11 on the body case 1 and in a direction of a second light guide means 72 disposed in the band 4. The first light guide means 71 is fitted into the opening portion 5 in a direction of the windbreaking glass 11 in such a manner that the opening portion 5 is filled with the first light guide means 71, which does not protrude from the body case 1.

Consequently, a light is emitted from the first light guide means 71 to the two directions of the windbreaking glass 11 and of the second light guide means 72.

The light emitting element 6 is disposed in the body case 1, and a light is emitted from the light emitting element 6 to the side of the first light guide means 71.

By such a configuration, a part of lights that have been emitted from the light emitting element 6 enters the first light guide means 71, and is guided to a direction of the windbreaking glass 11 via the first light guide means 71. The part of lights is then emitted upward from the opening portion 5 of the first light guide means 71.

Moreover, the other part of lights that have been emitted from the light emitting element 6 is guided from the opening portion 5 of the first light guide means 71 to a direction of the second light guide means 72 disposed in the band 4, and is emitted from the surface of the band 4.

Consequently, since lights that have been emitted from the light emitting element 6 are guided to the above both directions, a user can see a light emission from a part of the body case 1 and the band 4.

In the embodiment shown in FIG. 8, the first light guide means 71 is fitted into the opening portion 5 and is in a rectangular shape as shown in FIG. 8(b). However, a shape of the first light guide means 71 is not restricted to a rectangular shape.

Moreover, in place of forming the opening portion in the watch body, or in addition to forming the opening portion in the watch body, the opening portion 5 can be formed in a so-called bezel portion of the electronic wrist watch and the end face of the first light guide means 71 is exposed, thereby emitting a light from the periphery of the display device 2. By such a configuration, a further visibility effect and a decorative effect can be obtained.

### Ninth Embodiment

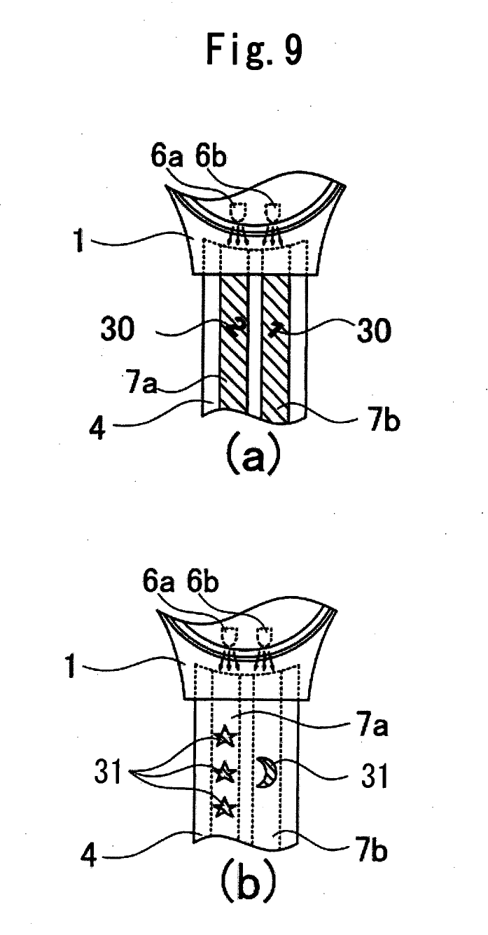

### FIG. 9

FIG. 9 is an elevation view illustrating an electronic wrist watch in accordance with a ninth embodiment of the present invention. In FIG. 9, a numeral 30 represents a shading member, and a numeral 31 represents an opening portion of the band 4.

In FIG. 9(a), the shading member 30 is disposed on a part of the surface of the light guide means 7a and 7b.

The shading member 30 is in a shape of a character, a symbol, or a figure. In the case in which a light enters the light guide means 7a and 7b, although the periphery of the shading member 30 is luminous, the section under the shading member 30 is shaded. Consequently, a shape of the shading member 30 can be conspicuous, and a high decorative effect can be obtained.

In the embodiment shown in FIG. 9(a), the shading members 30 are in shapes of characters of 1 and 2.

Moreover, by disposing a publicly known half mirror processing layer (not shown) on the surface of the light guide means 7a and 7b including the shading members 30, a shape of the shading member 30 can be visible only in the case in which a light enters the light guide means 7a and 7b. By such a configuration, a further decorative effect can be obtained.

In FIG. 9(b), the opening portion 31 is disposed on a part of the surface of the light guide means 7a and 7b. A light emitted from the light guide means 7a and 7b is visible via the opening portion 31.

The opening portion 31 is in a shape of a character, a symbol, or a figure. In the case in which a light enters the light guide means 7a and 7b, since the surface of the band 4 shades the periphery of the opening portion 31, only the opening portion 31 of the band 4 is luminous. Consequently, a pattern having a predetermined shape can be conspicuous by a light emission.

In the embodiment shown in FIG. 9(b), the opening portions 31 are in shapes of a crescent moon and a star.

By such a configuration, the band 4 can be luminous at one point in a predetermined shape or a predetermined pattern, thereby obtaining a high decorative effect.

Moreover, the opening portion 31 can be sealed with a transparent resin or a semitransparent resin, thereby improving a quality and decorativeness in a period in which a light emission is not carried out.

### Tenth Embodiment

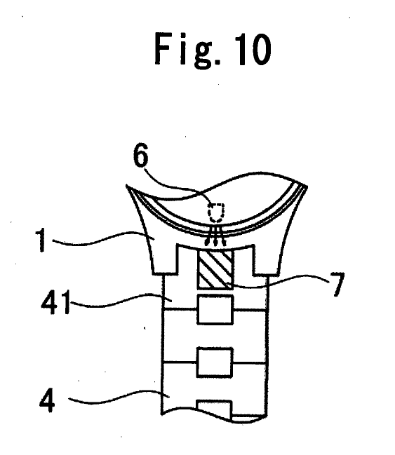

### FIG. 10

FIG. 10 is an elevation view illustrating an electronic wrist watch in accordance with a tenth embodiment of the present invention.

In FIG. 10, a numeral 41 represents a segment for configuring the band 4.

FIG. 10 shows the embodiment in which the light guide means 7 is disposed in the segment 41, which is an end portion connected with the body case 1, in the band 4 composed of a plurality of linked segments made of a metal or a resin member.

By such a configuration, a segment 41 different from the segment that includes the light guide means 7 can be removed or added, thereby easily adjusting a length of the band 4 while maintaining a light emitting function of the band 4.

Moreover, the light guide means 7 can be disposed in each of the segments although this is not shown in the figure. In such a case, the end portion of the light guide means 7 is disposed in an opening portion formed at the end of the segments abutted to each other in such a manner that the light guide means 7 of the plurality of segments are connected to each other for guiding a light. By such a configuration, the light that has been emitted from the light emitting element 6 can be guided even in the case in which a linking part for linking segments is disposed.

That is to say, even in the case in which a segment including the light guide means 7 is removed or added, alight emitting function of the band 4 is not obstructed, and a length of the band 4 can be easily adjusted while maintaining the light emitting function of the band 4.

Moreover, a light emitting pattern of the segments can be selected as needed. For instance, all segments can be luminous or every other segment can be luminous. By selecting the light emitting pattern, a wearing person can be informed of a variety of information.

### Embodiment 2

With reference to the block diagram, the following describes in detail a best mode in the case in which a worn type electronic device in accordance with the present invention is applied to a body-mounted type exercise training support apparatus as a biological measuring apparatus.

In the following embodiment, a worn type electronic device in accordance with the present invention is simply called a body-mounted type exercise training support apparatus. The following embodiment describes the case in which biological information is obtained by measuring a pulse wave as biological information.

In addition, the following embodiment describes the case in which the operations of the body-mounted type exercise training support apparatus are controlled by using a central processing unit (CPU).

Moreover, the following embodiment describes the case in which the body-mounted type exercise training support apparatus is a wrist watch type and is worn on a wrist of a user.

### Eleventh Embodiment

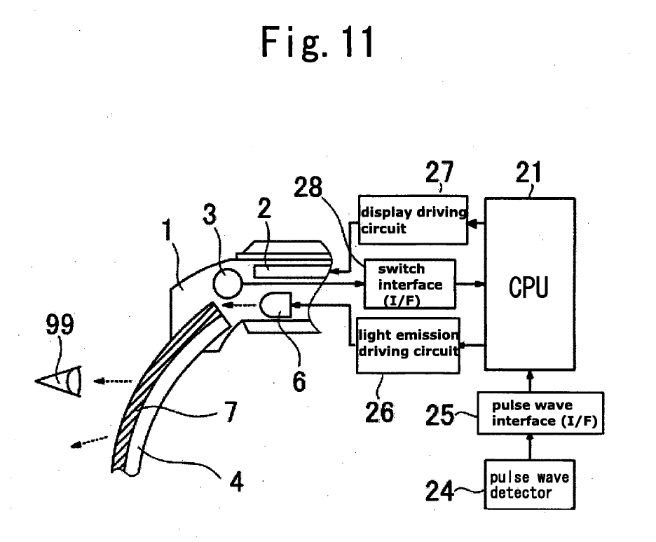

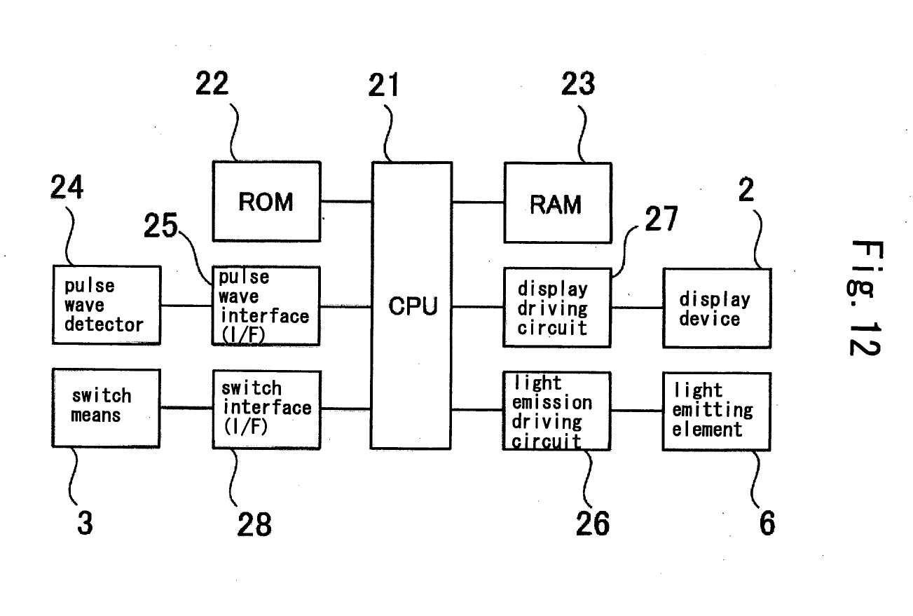

### FIGS. 11 and 12

FIG. 11 is a schematic side view showing the internal configuration of a body-mounted type exercise training support apparatus in accordance with an eleventh embodiment of the present invention, which is viewed from the side in a three o'clock direction.

In FIG. 11, a numeral 1 represents a body case, a numeral 2 represents a display device, a numeral 3 represents a switch means, a numeral 4 represents a band, a numeral 6 represents a light emitting means, a numeral 7 represents a light guide means, a numeral 21 represents a CPU, a numeral 24 represents a pulse wave detector, a numeral 25 represents a pulse wave interface (I/F), a numeral 26 represents a light emission driving circuit, a numeral 27 represents a display driving circuit, a numeral 28 represents a switch interface (I/F), and a numeral 99 represents a user, whose eye is shown schematically.

A dotted line arrow shows a route of a light and a full line arrow shows a flow of an electrical signal schematically.

FIG. 12 is a block diagram for illustrating an electric circuit of a body-mounted type exercise training support apparatus in accordance with the present invention.

In FIG. 12, a numeral 22 represents a read only memory (ROM), and a numeral 23 represents a random access memory (RAM).

FIG. 11 shows a schematic cross section of the body case 1, which is viewed from the side in a three or nine o'clock direction of a wrist watch, on the left side of the figure. The electrical connection to elements disposed in the body case 1 is shown on the right side of the figure by the circuit block diagram.

A first informing means is composed of the light emitting element 6 and the light guide means 7, and a second informing means is composed of the display device 2. The operations of the informing means are controlled by a CPU 21.

A switch means 3 is a means for inputting user information, and is composed of at least one switch. A user inputs user information by operating the switch means 3. The input user information is sent to the CPU 21 via the switch I/F 28.

The CPU 21 is an arithmetic processing unit that is a central section for controlling each circuit of the body-mounted type exercise training support apparatus in accordance with the present invention.

The ROM 22 stores processing programs that are executed by the CPU 21 and constants required for arithmetic processing or the like. The programs stored in the ROM 22 are described later.

The RAM 23 stores temporarily the results of the arithmetic processing executed by the CPU 21 and stores user information such as a user age and a resting heart rate.

Since it is troublesome to inputting user information every time the body-mounted type exercise training support apparatus in accordance with the present invention is used, it is preferable to hold the user information. Consequently, the RAM 23 can be composed of a nonvolatile storage device that is rewritable electrically.

The pulse wave detector 24 is a section for detecting a pulse wave of a user. Although it is not restricted in particular, there can be adopted an electrocardiogram detection system in a chest belt shape having an electrode to be worn on a chest for detecting a pulse wave, an optical system for detecting a pulse wave by a light in which an earlobe or a fingertip is caught between a light emitting element and a light intercepting element to measure a change of a blood flow, and a system using a pressure volume vibration method in which the pulsation of the heart of a living body is transmitted to a skin by an expansion and a contraction of a blood vessel and a surface vibration of the skin is detected.

The configuration and operation of the above systems are already known and the detailed descriptions thereof are omitted.

While the pulse wave detector 24 can be disposed in the body case 1 or the band 4, the pulse wave detector 24 can be formed separately from the body case 1 and the band 4. In such a case, the pulse wave detector 24 and the CPU 21 transmit and receive signals with each other by a wire or radio transmission.

The pulse wave interface (I/F) 25 receives an analog output signal of the pulse wave detector 24 at a specified interval and converts the analog signal to a digital signal for an output. In the case in which an output signal from the pulse wave detector 24 is transmitted by a radio, a receiving circuit is included in the pulse wave interface (I/F) 25 for receiving the output signal although this is not shown in the figure.

The light emission driving circuit 26 controls an electric current that flows to the light emitting element 6 corresponding to the control data output from the CPU 21. The electric current control can be an analog type or a digital type based on the time shared driving.

The light emitting element 6 is disposed in the body case 1 and the light guide means 7 is disposed in the band 4. By such a configuration, the light that has been emitted from the light emitting element 6 enters the light guide means 7 disposed in the band 4.

The light guide means 7 diffuses the incident light to the outside in a direction of a thickness of the band 4 in such a manner that the light becomes visible for a user. By such a configuration, a light can be emitted from the band 4 even in the case in which a light emitting element is not disposed in the band 4.

As a matter of course, an opening portion (not shown) can be formed in the body case 1, and the light guide means 7 can be fitted into the opening portion in such a manner that the opening portion 5 in a direction of the windbreaking glass 11 is filled with the light guide means like the embodiment shown in FIG. 8, thereby diffusing the light that has been emitted from the light emitting element 6 via the opening portion.

By such a configuration, a light can be emitted from a part of the body case 1.

A first informing means is composed of the light emitting element 6 and the light guide means 7. A plurality of light guide means 7 can also be formed. By such a configuration, more lights that have been emitted from the light emitting element 6 can be transmitted to the band 4, and a light emission from the band 4 can be more vivid.

The light emission state of the band 4 can be determined corresponding to a shape of the light guide means. Consequently, even in the case in which a light emission area of the band 4 is increased, the power consumption of the present apparatus is not increased.

The band 4 and the body case 1 can be connected to each other by a band fitting structure having become common property for a wrist watch. Consequently, the detailed descriptions of the structure are omitted.

The display driving circuit 27 receives the display information from the CPU 21 and drives the display device 2 by converting the information to the electric signal of the type required for the display device 2 and outputting the signal.

The switch interface (I/F) 28 converts the user information that has been input by operating the switch means 3 to an electric signal of a type that can be received by the CPU 21 for an output. The CPU 21 receives the electric signal and stores the signal in the RAM 23 as the user information.

The display device 2 is a second informing means, which displays a heart rate, a training index, time, user information, and device information.

The first informing means composed of the light emitting element 6 and the light guide means 7 can inform of support information and apparatus information from the body-mounted type exercise training support apparatus in accordance with the present invention even during exercise for a user, and has a role for strongly attracting user attention. On the other hand, the second informing means is for informing of more detailed information.

As the display device 2, for instance, a liquid crystal display device can be used. The liquid crystal display device can display characters or pictographs, and is suitable for a lightweight display device that consumes low power. The liquid crystal display device can also implement a color display and a graphics display adopting a dot matrix.

Moreover, as the display device 2, a liquid crystal display device having a memory function such as a ferroelectric liquid crystal display device can also be used. The liquid crystal display device having a memory function requires power only in switching a display, and holds the state of the switched display, in which the hold of the state requires no power consumption. Consequently, the display device 2 consumes no power during a display, thereby implementing a low power consumption device.

As a matter of course, the display device 2 can also be so-called a dial of an analog display type, which informs of time by watch hands like an analog display type watch.

The CPU 21 detects a pulse wave from a digital signal output from the pulse wave interface (I/F) 25, and calculates a heart rate of a user by a heart rate calculation means.

As described before, a heart rate is generally a value of heart beats per one minute. Although a method of calculating a heart rate is not restricted in particular, there are mentioned, for instance, a method in which pulse waves are detected for ten seconds and the detected value of the pulse wave is multiplied by six to obtain heart beats per one minute, and a method in which pulse waves of a living body is detected in a certain period to obtain the average period of the pulse waves and one minute is divided by the average period to obtain a heart rate.

The heart rate calculation means uses the above methods and the operation programs thereof are stored in the ROM 22. The CPU 21 reads and executes the operation programs.

The switch means 3 is electrically connected to the CPU 21 via the switch interface (I/F) 28. As described before, the switch interface (I/F) 28 converts an operation of the switch means 3 to an electric signal that can be used by the CPU 21 and outputs the signal.

By user's operation of the switch means 3, user information such as an age of a user, a resting heart rate, a target heart rate, and a target exercise intensity can be input. In addition, the function of the body-mounted type exercise training support apparatus in accordance with the present invention can be selected, and the function can be started, stopped, activated, or inactivated by an indication. For instance, a light emitting position, a light emitting condition, and a light emitting pattern of the light emitting element 6 can be modified.

A plurality of character strings and functions are assigned to the switch means 3 corresponding to an operation program stored in the ROM 22, and the character strings and functions are input by operating the switch means 3. The configuration in which a plurality of character strings and so on are assigned to one switch to input information by the operation thereof is widely used for an input means of a compact information device such as a portable telephone and personal digital assistants (PDA), and the detailed descriptions thereof are omitted.

It is preferable to prepare a plurality of switch means 3. A size and a shape of the switch means 3 can be varied corresponding to a function assigned to each switch means 3. By such a configuration, a further improvement of an operational property can be expected.

The CPU 21 calculates a target heart rate based on user information that is input in advance using the switch means 3 as a user information input means. In the case in which a target heart rate has been input as user information, the input target heart rate is used as a target heart rate that has been already set.

By using a comparison operating means, the CPU 21 compares a target heart rate calculated from the input user information and a heart rate calculated from biological information that has been measured. The comparison operating means is stored as an operation program in the ROM 22. The CPU 21 reads and executes the operation program.

Support information such as the comparison results that have been obtained by the comparison operating means and a heart rate that has been measured are stored in the RAM 23 and informed of by the informing means as needed.

The informing means is provided with the first informing means and the second informing means. A light emission of the light emitting element 6 and the light guide means 7 that are the first informing means is controlled to emit a light from the band 4 for information, and the display device 2 as the second informing means displays the details using characters and pictographs for information.

As described above, support information that will be a training index is generated based on information that is obtained from a pulse wave, and information is transmitted to a user by using the two informing means.

While the above description uses a heart rate as a criterion, exercise intensity can also be used as a criterion by a reverse operation.

The first informing means and the second informing means can update support information at predetermined periods, respectively. As a matter of course, the first informing means and the second informing means can be made synchronous or asynchronous to update and inform of support information.

An update of support information is to rewrite information displayed on the display device 2 as the second informing means at a constant interval.

Even in the case in which the display driving circuit 27 malfunctions by a static electricity or magnetism applied from the outside and a character string that does not make any sense or the like is displayed on the display device 2, information to be displayed on the display device 2 can be almost forcibly rewritten at a predetermined period by an indication from the CPU 21. Therefore, the display device 2 can modify an error display. The predetermined period is, for instance, 1 second although it is not restricted in particular.

Moreover, support information can be updated and informed of in the case in which a change in a measured heart rate is detected or a change in the comparison result between a measured heart rate and a target heart rate is detected.

Furthermore, support information can be updated and informed of at a predetermined period, and support information can also be updated and informed of immediately when a change in a measured heart rate is detected or a change in the comparison result between a measured heart rate and a target heart rate is detected.

As described before, a liquid crystal display device having a memory function can be used as the display device 2.

In the case in which a liquid crystal display device having a memory function is used, displayed information is held until the information or the displayed content is modified. Therefore, a futile rewrite of information can be reduced by discontinuing rewriting after a predetermined period. By such a configuration, power consumption pertaining to a display can be further reduced.

As described above, by updating support information, while immediate informing of a training index by the first informing means is maintained, an increase in power consumption pertaining to an information display by the second informing means can be suppressed.

The first informing means and the second informing means can inform of a plurality of support information in the time sharing manner.

The light emitting element 6 can be controlled synchronously with a pulse wave that is detected. For instance, it can be displayed that the apparatus is correctly worn and operated by controlling the light emitting element 6 synchronously with a pulse wave for a predetermined time or until a predetermined pulse wave is detected after a measurement is started.

By such a configuration, a user can confirm that the measurement has been started correctly, thereby giving peace of mind to the user.

On the contrary, in the case in which a state in which a pulse wave cannot be detected continues for a predetermined time, the light emitting element 6 can be controlled to stimulate a user to detach the apparatus and correctly wear the apparatus again, or user's detaching of the apparatus can be judged and the measurement can be suspended, thereby suppressing an increase in power consumption.

As a matter of course, information for stimulating a user to correctly wear the apparatus again and a suspension of a measurement can be processed in order.

Information using the light emitting element 6 can be displayed by a variation in a light emission such as a fast blink in the state of overtraining (excess training), a slow blink in the state of under-training (insufficient training), and lights-out in the state of target training. In addition, information can be displayed by a variation in a luminance of the light emitting element 6, a variation in a luminous color of the light emitting element 6, or a selection of a light emission from a plurality of the light emitting elements 6.

As a matter of course, any of the above displays can be combined, and information other than a training index can also be informed of.

For instance, the first informing means can display information of the body-mounted type exercise training support apparatus in accordance with the present invention.

More specifically, the light emitting element 6 can be controlled based on information from a residual battery capacity detection means (not shown) to inform of a residual capacity of a battery for a predetermined time. As a matter of course, a control of the light emitting element 6 based on the residual battery capacity detection means can be activated or inactivated by operating the switch means 3.

As the residual battery capacity detection means, for instance, a battery voltage is detected by an A/D converter to estimate a residual capacity of the battery. By such a configuration, in the case in which an extreme reduction in a residual capacity of the battery is detected, a lighting time of the light emitting element 6 can be made shorter than a predetermined time by a control of the light emitting element 6, or a light emission of the light emitting element 6 can be inactivated, thereby suppressing an increase in power consumption and increasing an operating time of the body-mounted type exercise training support apparatus in accordance with the present invention.

In the case of a state that the battery has little residual capacity or an abnormal state of the apparatus such as an interruption of an operation of a part of the system, the state can be informed of. As the information, the light emitting element 6 can be controlled to inform of an abnormal state of the apparatus for a predetermined time.

As a matter of course, a control of the light emitting element 6 based on an abnormal state of the apparatus can be activated or inactivated by operating the switch means 3.

As a detection means of an abnormal state, an abnormal state can be detected by finding that a predetermined response of a circuit is suspended for a predetermined time.

The information of an abnormal state of the apparatus must be different from that of a normal state of the apparatus. For instance, a regularly repeated blink can be generated at a period that is abnormal as a heart rate such as 30 blinks or less per minute or 220 blinks or larger per minute, or at a period of an irregular blink that is apparently different from a pulse.

As described before, information from the first informing means composed of the light emitting element 6 and the light guide means 7 having a particularly excellent visibility can be made to have a highly important meaning, thereby enabling a user to quickly modify an exercise training and to deal with a problem caused by the state of an apparatus.

However, a user may forget a meaning of information from the first informing means in some cases. For instance, since a reduction of a residual battery capacity does not frequently occur, a user hardly ever sees the information pattern. Consequently, even in the case in which a user confirms that some change has occurred by the information, the user may not understand the meaning.

Accordingly, the details of the information contents of the first informing means are also displayed on the display device 2 that is the second informing means. By such a configuration, even in the case in which a user is not used to the body-mounted type exercise training support apparatus in accordance with the present invention, the user can confirm information from the light emitting element 6, the meaning thereof, and the relationship by seeing the display device 2, thereby assisting the user with utilizing the body-mounted type exercise training support apparatus in accordance with the present invention.

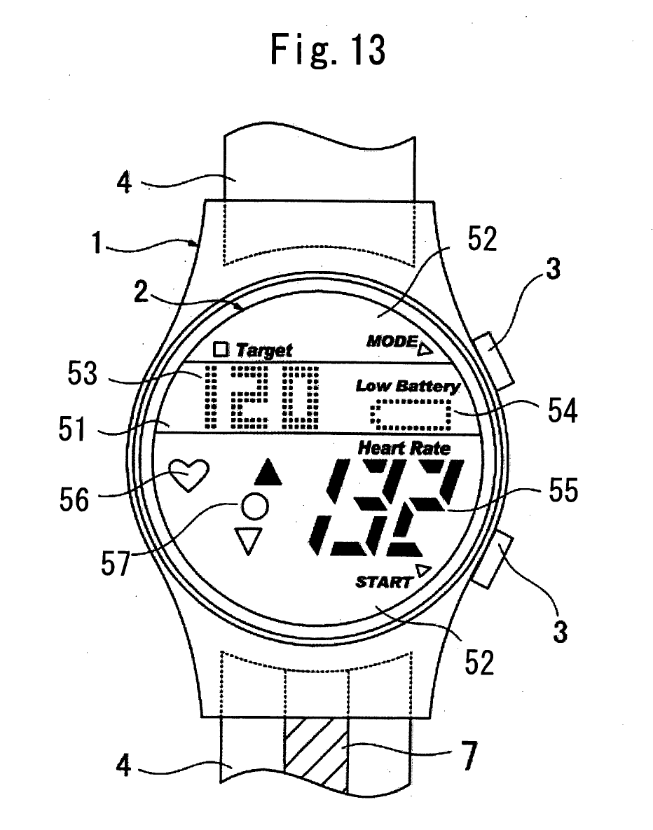

FIG. 13 is an elevation view illustrating an embodiment in which information is displayed on a display device 2 that is a second informing means.

In FIG. 13, a numeral 51 represents a dot matrix display area, a numeral 52 represents a segment display area, and numerals 56 and 57 represent icons (pictographs).

Here, elements equivalent to those illustrated previously are numerically numbered similarly.

The dot matrix display area 51 displays a target heart rate and a residual capacity of a mounted battery. The segment display area 52 displays a heart rate that is currently measured.

In the embodiment shown in FIG. 3, a target heart rate display 53 indicates a heart rate of 120 and a current heart rate display 55 indicates a heart rate of 132. A residual battery capacity display 54 shows an empty pattern indicating a battery and displays “Low Battery” that indicates that the battery has little residual capacity.

The icon 57 indicates that a current heart rate is higher than a target heart rate that has been input and set. The icon 56 is a display that indicates that a pulse wave is being correctly detected by blinking synchronously with the pulse wave.

As a matter of course, a display pattern of the body-mounted type exercise training support apparatus in accordance with the present invention is not restricted to the present embodiment. As described above, however, a light is used for the first informing means and the characters and pictographs are used for the second informing means, thereby quickly informing a user of required information.

While the above embodiment describes the case in which the first informing means and the second informing means inform of almost identical information contents or overlapped information contents, the present invention is not restricted to the above embodiment as a matter of course. For instance, the light emitting element 6 can be used to stimulate a user to see the display device 2 as a trigger of information to the user. As an example, the first informing means can be blinked intensively.

In such a case, the information contents of the first informing means can be different from those of the second informing means. For instance, in the case in which a target heart rate is almost reached during exercise, the first informing means informs of the state by a light emitting pattern, and the second informing means displays a current heart rate that is being measured.

A shape of the first informing means composed of the light emitting element 6 and the light guide means 7 in the body-mounted type exercise training support apparatus in accordance with the present invention is not restricted to the embodiment shown in FIG. 11, and can be selected as needed like the above described embodiments shown in FIGS. 1 to 10 for instance.

The light emitting element 6 can also be used as a back light that illuminates the display device 2 from the back face although this is not shown in the figure.

Moreover, the body-mounted type exercise training support apparatus in accordance with the present invention can inform of a training index for a predetermined time by using a signal that is input from a sensing means (not shown) as a trigger. In such a case, the sensing means can be activated or inactivated by the switch means 3.

As the sensing means, there are mentioned, for instance, a vibration sensor for outputting a signal by a detection of a vibration, an inclination sensor for outputting a signal by a detection of an inclination, an acceleration sensor for outputting a signal by a detection of an acceleration, a temperature sensor, an atmospheric pressure sensor, and a sensor for outputting a signal by a detection of receiving of a radio wave.

The sensing means can also have a role of the switch means 3. More specifically, a user can strongly shake or hit an arm on which the body-mounted type exercise training support apparatus in accordance with the present invention has been worn to temporarily operate a control of the light emitting element 6 that is used for informing of a training index without operating the switch means 3. By such a configuration, power consumption can be reduced and a user can be released from a troublesome switching operation. The sensing means is useful particularly during exercise.

The above described body-mounted type exercise training support apparatus in accordance with the present invention can improve a visibility and a variety in a representation due to a light emission of the band 4, a wearing characteristic as an apparatus, a sufficient operating time, and a fashionable property without enlarging the mounted display device 2 to more than necessity.

### Embodiment 3

### Twelfth Embodiment

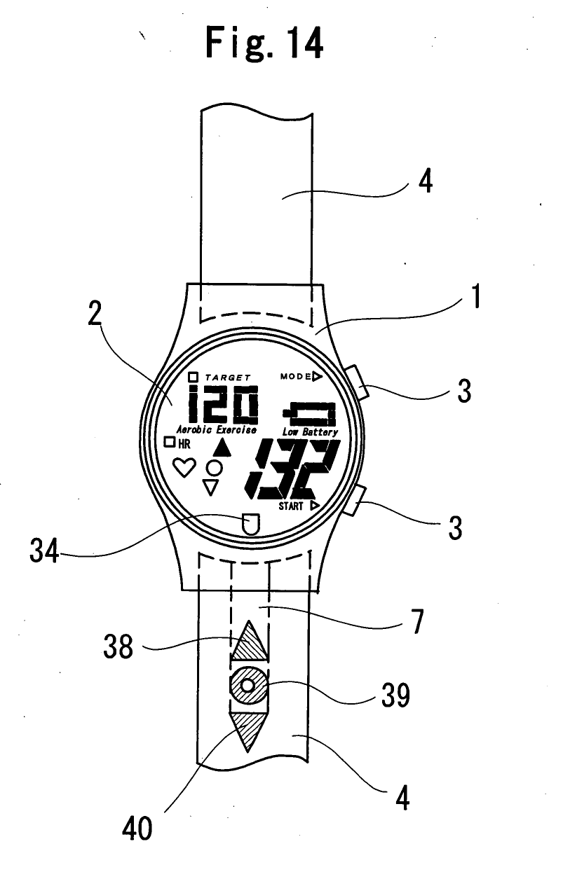

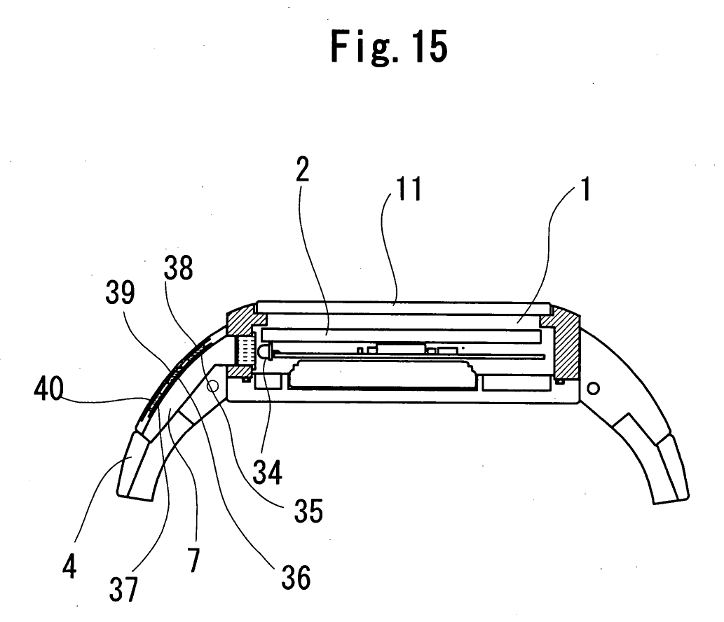

### FIGS. 14 and 15

FIG. 14 is an elevation view illustrating an electronic wrist watch in accordance with an eleventh embodiment of the present invention. FIG. 15 is a schematic side view showing the internal configuration of the electronic wrist watch shown in FIG. 14, which is viewed from the side in a three o'clock direction.

The electronic wrist watch in accordance with the present embodiment has a configuration basically similar to that of the embodiment shown in FIG. 1. Therefore, elements equivalent to those illustrated in FIG. 1 are numerically numbered similarly and the detailed descriptions of the equivalent elements are omitted.

The electronic wrist watch in accordance with the present embodiment is provided with an LED light emitting element 34 composed of three color LEDs that are a red (R) LED light emitting element, a green (G) LED light emitting element, and a blue (B) LED light emitting element in the body case 1.

The LED light emitting element 34 can selectively emit lights having wave lengths of three colors of red (R), green (G), and blue (B) by a control of a control device (not shown) disposed in the body case 1.

As shown in FIGS. 14 and 15, the light guide means 7 is disposed in the band 4. Moreover, the band 4 is provided with a red (R) filter 35 which only a red (R) light emitted from the red (R) LED light emitting element of the LED light emitting element 34 can pass through, a green (G) filter 36 which only a green (G) light emitted from the green (G) LED light emitting element of the LED light emitting element 34 can pass through, and a blue (B) filter 37 which only a blue (B) light emitted from the blue (B) LED light emitting element of the LED light emitting element 34 can pass through, on the band surface side of the light guide means 7.

A red (R) index portion 38, a green (G) index portion 39, and a blue (B) index portion 40 are formed on the upper face (on the surface side of the band 4) of the red (R) filter 35, the green (G) filter 36, and the blue (B) filter 37, respectively, and can be luminous by the red (R) light, the green (G) light, and the blue (B) light that have passed through the filters 35, 36, and 37, respectively.

As described above, the index portions 38, 39, and 40 are formed on the surface of the filters 35, 36, and 37, and are made to be luminous by a light emitted from the LED light emitting element 34, in which the light has a predetermined wave length and passes through the filter 35, 36, or 37. A luminous color and a light emitting position can be freely selected by modifying a shape, a type, and a disposed position of the index portions. Consequently, a light emitting position and a light emitting pattern can be freely produced, thereby improving a visibility and a fashionable property due to a light emission.

In the above embodiment, the LED light emitting element 34 composed of three color LEDs that are a red (R) LED light emitting element, a green (G) LED light emitting element, and a blue (B) LED light emitting element is disposed in the body case 1. As a matter of course, the LED light emitting element 34 can also be a color LED that can emit a red (R) light, a green (G) light, and a blue (B) light by controlling a driving electric current to be applied.

Moreover, a red (R) LED light emitting element, a green (G) LED light emitting element, and a blue (B) LED light emitting element that are separately formed can also be disposed as a matter of course.

In such a case, a light can be emitted separately from the three color LEDs of the red (R) LED light emitting element, the green (G) LED light emitting element, and the blue (B) LED light emitting element of the LED light emitting element 34, and the index portions 38, 39, and 40 can be separately made to be luminous. Moreover, the luminous index portions 38, 39, and 40 can also be selected by modifying the combination of light emissions of the LEDs.

Moreover, as a matter of course, a light can be emitted from all of the three color LEDs of the red (R) LED light emitting element, the green (G) LED light emitting element, and the blue (B) LED light emitting element of the LED light emitting element 34, and all of the index portions 38, 39, and 40 can be made to be luminous.

Even in the case of the present embodiment, a disposition and a shape of the light guide means 7 and the index portion are not restricted to the present embodiment, and can be selected as needed like the above described embodiments shown in FIGS. 1 to 10 for instance.

### Thirteenth Embodiment

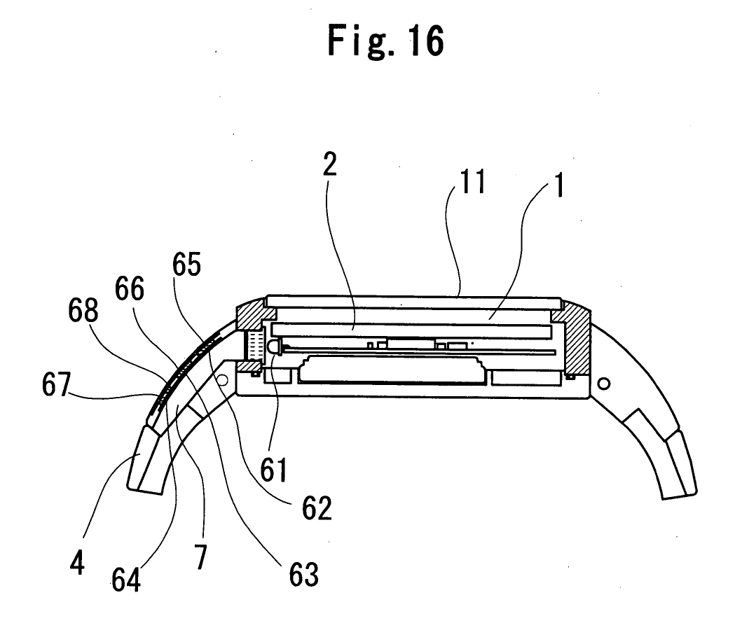

### FIG. 16

FIG. 16 is a schematic side view illustrating an electronic wrist watch in accordance with a thirteenth embodiment of the present invention and showing the internal configuration of the electronic wrist watch, which is viewed from the side in a three o'clock direction.

The electronic wrist watch in accordance with the present embodiment has a configuration basically similar to that shown in FIGS. 14 and 15. Therefore, elements equivalent to those illustrated in FIGS. 14 and 15 are numerically numbered similarly and the detailed descriptions of the equivalent elements are omitted.

The electronic wrist watch in accordance with the present embodiment is provided with a plurality of ultraviolet light emitting elements (UV-LED) 61 in the body case 1 as shown in FIG. 16.

The ultraviolet light emitting elements 61 can separately emit ultraviolet lights having different wave length widths by a control of a control device (not shown) disposed in the body case 1.

As shown in FIG. 16, the light guide means 7 is disposed in the band 4. Moreover, the band 4 is provided with band-pass filters 62, 63, and 64 on the band surface side of the light guide means 7 corresponding to the ultraviolet light emitting elements 61. The band-pass filters 62, 63, and 64 can select ultraviolet lights that have different wave length widths and that have been emitted from the ultraviolet light emitting elements 61 and can make the selected ultraviolet lights pass through the filters.

The index portions 65, 66, and 67 are formed on the upper face of the band-pass filters 62, 63, and 64, respectively. The index portions 65, 66, and 67 are made of fluorescent substances that emit lights selectively by the ultraviolet lights that have different wave length widths and that have passed through the band-pass filters 62, 63, and 64.

Moreover, an ultraviolet filter 68 is formed on the upper face (on the surface side of the band) of the index portions 65, 66, and 67. The ultraviolet filter 68 can cut an ultraviolet light having a wave length of 400 nm or less, for instance, in order to prevent a light emission of the index portions 65, 66, and 67 due to an external ultraviolet light.

As described above, in the case in which the light emitting element 6 is an ultraviolet light emitting element (UV-LED) 61, the band-pass filters 62, 63, and 64 are disposed to select ultraviolet lights that have different wave lengths and that have been emitted from the ultraviolet light emitting elements 61 and to make the selected ultraviolet lights pass through the filters. In addition, since a fluorescent substance that emits a light having a predetermined color by an ultraviolet light is disposed, a light emitting position and a light emitting shape can be selected, thereby improving a visibility and a fashionable property due to a light emission.

In such a case, a wave length of an ultraviolet light emitted from the ultraviolet light emitting element 61 can be selected to separately emit the light, and the index portions 65, 66, and 67 can be separately made to be luminous. Moreover, the luminous index portions 65, 66, and 67 can also be selected by modifying the combination of light emissions of the ultraviolet light emitting elements 61.

Moreover, as a matter of course, a light can be emitted from all of the ultraviolet light emitting elements 61, and all of the index portions 65, 66, and 67 can be made to be luminous.

While the present embodiment adopts the ultraviolet light emitting element (UV-LED) 61 as the light emitting element 6, a light emitting element having a wave length of a light other than an ultraviolet light, such as an electron beam emitting element, can also be used as the light emitting element 6 as a matter of course.

Even in the case of the present embodiment, a disposition and a shape of the light guide means 7 and the index portion are not restricted to the present embodiment, and can be selected as needed like the above described embodiments shown in FIGS. 1 to 10 for instance.

### Fourteenth Embodiment

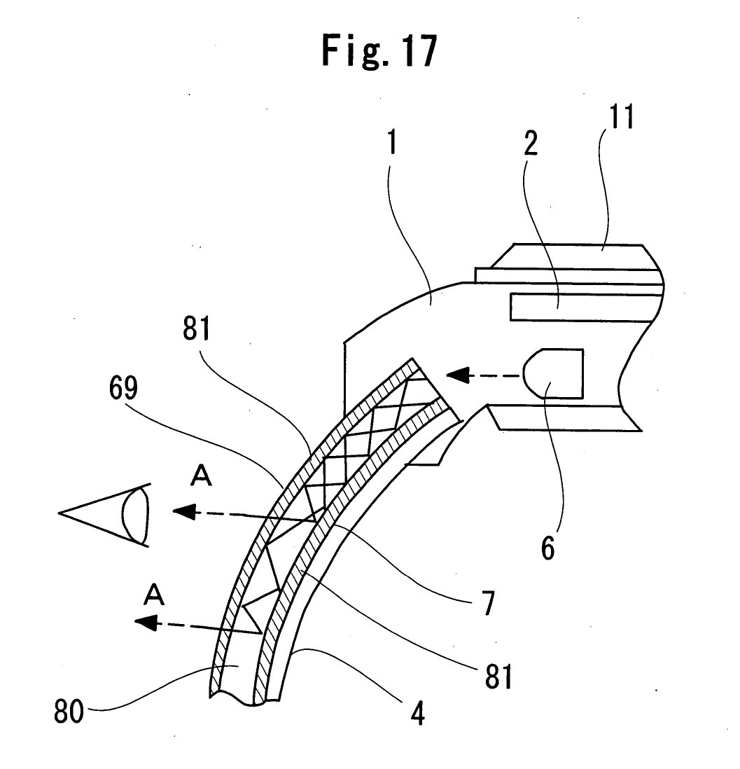

### FIG. 17

FIG. 17 is a schematic side view illustrating an electronic wrist watch in accordance with a fourteenth embodiment of the present invention and showing the internal configuration of the electronic wrist watch, which is viewed from the side in a three o'clock direction.

The electronic wrist watch in accordance with the present embodiment has a configuration basically similar to that of the embodiment shown in FIG. 1. Therefore, elements equivalent to those illustrated in FIG. 1 are numerically numbered similarly and the detailed descriptions of the equivalent elements are omitted.

In the electronic wrist watch in accordance with the present embodiment, the light guide means 7 disposed in the band 4 is composed of an optical fiber 69 as shown in FIG. 17. The optical fiber 69 has a two-layer structure composed of a central core 80 that has a high refractive index and that is made of a quartz glass or a plastic and a clad 81 that has a low refractive index and that covers the core. The band 4 is formed in a straight state in a design. In the case in which the band 4 is worn on a wrist to be bent and an incident angle of a light exceeds a critical angle of a reflection, the light is emitted from the clad 81 on the surface side of the band 4 as shown by an arrow A.

By such a configuration, the light that has been emitted from the light emitting element 6 disposed in the body case 1 can be reliably guided via the optical fiber 69 and can be reliably emitted from the surface of the band 4. In addition, the structure of the band 4 can be prevented from being complicated, and a visibility and a fashionable property due to a light emission of a band 4 can be improved.

Even in the case of the present embodiment, a disposition and a shape of the light guide means 7 and the index portion are not restricted to the present embodiment, and can be selected as needed like the above described embodiments shown in FIGS. 1 to 10 for instance.

### Fifteenth Embodiment

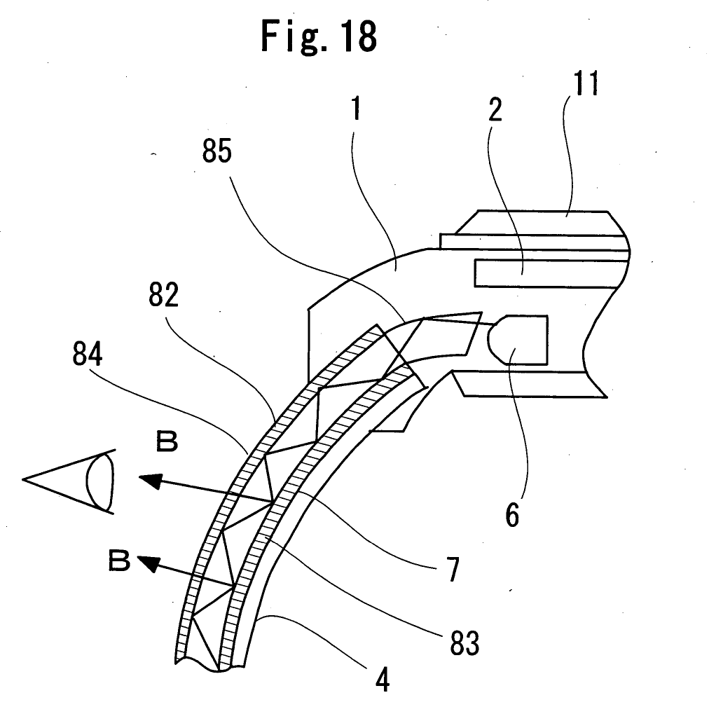

### FIG. 18

FIG. 18 is a schematic side view illustrating an electronic wrist watch in accordance with a fifteenth embodiment of the present invention and showing the internal configuration of the electronic wrist watch, which is viewed from the side in a three o'clock direction.

The electronic wrist watch in accordance with the present embodiment has a configuration basically similar to that of the embodiment shown in FIG. 1. Therefore, elements equivalent to those illustrated in FIG. 1 are numerically numbered similarly and the detailed descriptions of the equivalent elements are omitted.

In the electronic wrist watch in accordance with the present embodiment, the light guide means 7 disposed in the band 4 is composed of a light guide member 84 provided with diffraction gratings (holograms) 82 and 83 on the both faces thereof as shown in FIG. 18.

An incident prism 85 is formed on the light emitting element 6 side of the light guide member 84. The incident prism 85 can be fabricated by being integrated with the light guide member 84 or by being made of a simple substance of a prism and being bonded to the light guide member 84.

The diffraction gratings (holograms) 82 and 83 can diffract a light that has a predetermined wave length and that has entered at a predetermined angle, and can emit the light at a predetermined angle as shown by an arrow B in FIG. 18.

Consequently, in the case in which the light guide member 84 is disposed in the entire or a part of the band 4, a light can be selectively emitted from the entire or a part of the band.

Moreover, a shape of the diffraction grating (hologram) 82 on the surface of the band 4 can be a character or a picture, thereby improving a visibility and a fashionable property.

In such a case, by using a white light as a light emitted from the light emitting element 6, a color of a light emitted from the diffraction grating 82 is varied depending on a view angle, thereby improving a visibility and a fashionable property.

By such a configuration, the light that has been emitted from the light emitting element 6 disposed in the body case 1 can be reliably guided by the light guide member 84 provided with the diffraction gratings 82 and 83 on the both faces thereof, and can be diffracted at a gap of a slit of the diffraction grating to reliably emit the light from the surface of the band 4. In addition, the structure of the band 4 can be prevented from being complicated, and a visibility and a fashionable property due to a light emission of a band 4 can be improved.

Even in the case of the present embodiment, a disposition and a shape of the light guide means 7 and the index portion are not restricted to the present embodiment, and can be selected as needed like the above described embodiments shown in FIGS. 1 to 10 for instance.

### Sixteenth Embodiment

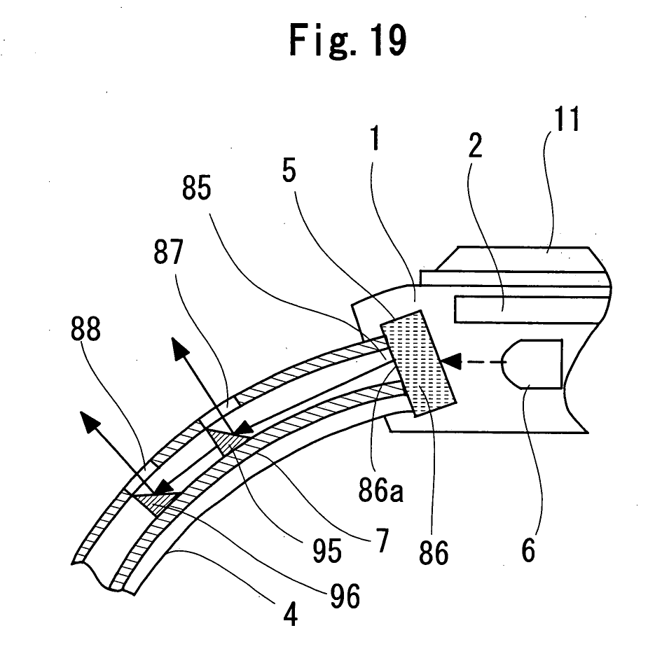

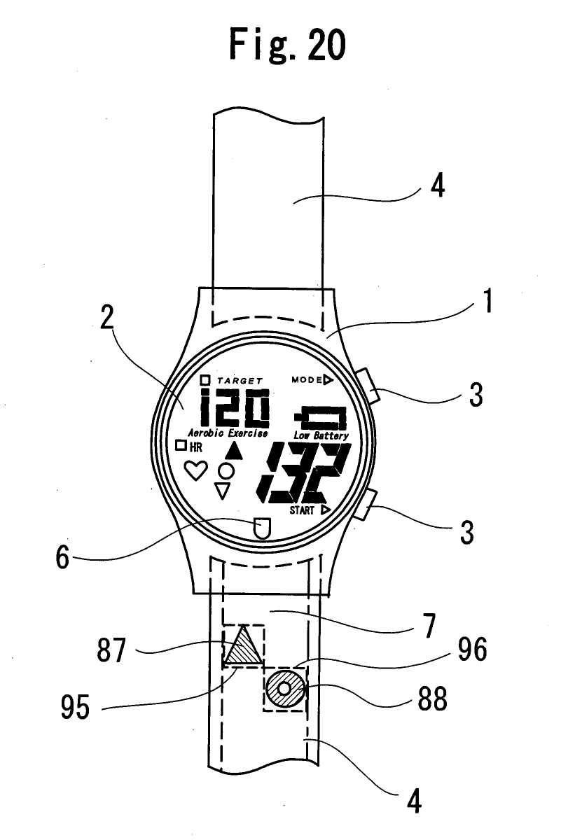

### FIGS. 19 and 20

FIG. 19 is a schematic side view illustrating an electronic wrist watch in accordance with a sixteenth embodiment of the present invention and showing the internal configuration of the electronic wrist watch, which is viewed from the side in a three o'clock direction. FIG. 20 is an elevation view of the electronic wrist watch shown in FIG. 19.

The electronic wrist watch in accordance with the present embodiment has a configuration basically similar to that of the embodiment shown in FIG. 1. Therefore, elements equivalent to those illustrated in FIG. 1 are numerically numbered similarly and the detailed descriptions of the equivalent elements are omitted.

The electronic wrist watch in accordance with the present embodiment is provided with a liquid crystal element 86 in the opening portion 5 of the body case 1 as shown in FIG. 19.

As shown in FIGS. 19 and 20, the band 4 includes a plurality of index deflection members having deflection angles intersecting with each other at right angles on the band surface side of the light guide means 7, wherein two index deflection members 87 and 88 are formed in the present embodiment.

As shown in FIG. 20, the index deflection members 87 and 88 are shifted to each other in the transverse direction of the band 4 to be disposed in such a manner that the optical paths for the index deflection members 87 and 88 are not obstructed by each other.

Moreover, the light guide means 7 contains optical path modification means 95 and 96 such as a mirror and a prism under the index deflection members 87 and 88 disposed being shifted to each other in the transverse direction of the band 4. By such a configuration, as shown by arrows in FIG. 19, the light that has been emitted from the light emitting element 6 disposed in the body case 1 passes through the liquid crystal element 86 and is guided to the light guide means 7 on the band 4 side. The direction of the light is then turned to the index deflection members 87 and 88 by the optical path modification means 95 and 96.

The plurality of index deflection members 87 and 88 that configure an index portion can be selected to diffuse the light by varying a voltage to be applied to the liquid crystal element 86 by using a control device (not shown) disposed in the body case 1.

By such a configuration, the light that has been emitted from the light emitting element 6 disposed in the body case 1 passes through the liquid crystal element 86 and is guided to the light guide means 7 on the band 4 side.

By varying a voltage to be applied to the liquid crystal element 86, a light that has passed through the liquid crystal element 86 can have a predetermined deflection angle. Consequently, a light can be emitted from the index deflection member 87 or 88 that has a deflection angle corresponding to the above deflection angle and from the surface of the band 4.

More specifically, although this is not shown in the figure, for instance, an index deflection member that makes a light having horizontal amplitude pass through the member is disposed between the light emitting element 6 and the liquid crystal element 86 that are disposed in the body case 1, and a voltage to be applied to the liquid crystal element 86 is controlled to activate a 90-degrees deflection (ON control).

By such a configuration, a light that has passed through the liquid crystal element 86 is changed to a light deflected in a vertical direction. In the case in which the index deflection member 87 is an index deflection member that makes a light with horizontal amplitude pass through the member, the light that has passed through the liquid crystal element 86 and that has been changed to a light deflected in a vertical direction is cut off.

On the other hand, in the case in which the index deflection member 88 is an index deflection member that makes a light with vertical amplitude pass through the member, a light that has passed through the liquid crystal element 86 and that has been changed to a light deflected in a vertical direction is made to pass through the member.

Moreover, a voltage to be applied to the liquid crystal element 86 can be controlled to inactivate a 90-degrees deflection (OFF control). By such a configuration, in the case in which a light having horizontal amplitude passes through the member and the index deflection member 87 is an index deflection member that makes a light with horizontal amplitude pass through the member, a light that has passed through the liquid crystal element 86 and that has horizontal amplitude is made to pass through the member.

On the other hand, in the case in which the index deflection member 88 is an index deflection member that makes a light with vertical amplitude pass through the member, a light that has passed through the liquid crystal element 86 and that has horizontal amplitude is cut off.

In such a case, the liquid crystal element 86 can be a publicly known segment liquid crystal panel, a dot matrix liquid crystal panel, or a liquid crystal panel provided with a pattern such as a picture, a character, and a symbol on the surface 86a on the band 4 side of the liquid crystal element 86. For instance, a blink and a liquid crystal pattern can be modified based on the control of the CPU 21.

That is to say, a dot matrix liquid crystal pattern can be modified as needed, and the pattern is not restricted in particular. Although this is not shown in the figure, for instance, the pattern can be a full transmission pattern, a transmission pattern of a spot pattern, a transmission pattern of a stripe pattern (a vertical stripe, a horizontal stripe, or a diagonal stripe), and a pattern that restricts a transmission area. Any of the pattern areas can be selected to be blinked.

Consequently, a light emitting position and a light emitting shape can be selected only by varying a voltage to be applied to the liquid crystal element 86, thereby improving a visibility and a fashionable property due to a light emission. In addition, the structure of the band 4 can be prevented from being complicated, and a visibility and a fashionable property due to a light emission of a band 4 can be improved.

Even in the case of the present embodiment, a disposition and a shape of the light guide means 7 and the index portion are not restricted to the present embodiment, and can be selected as needed like the above described embodiments shown in FIGS. 1 to 10 for instance.

### Seventeenth Embodiment

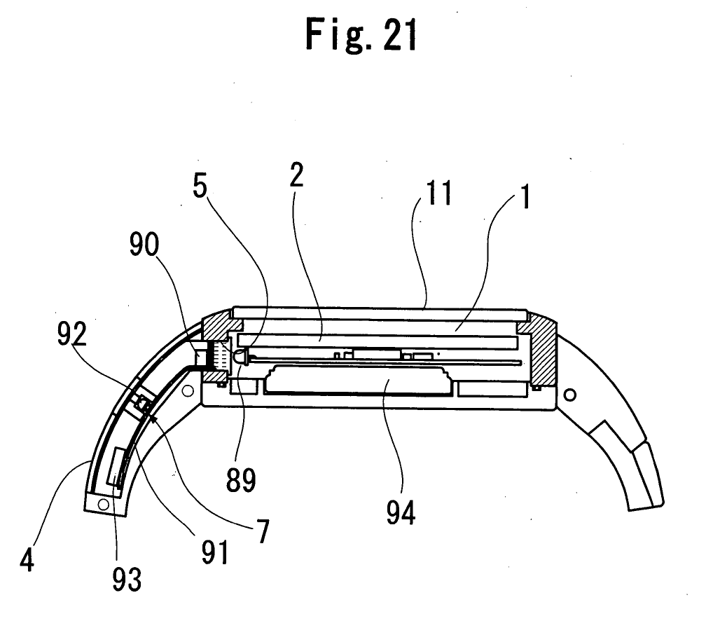

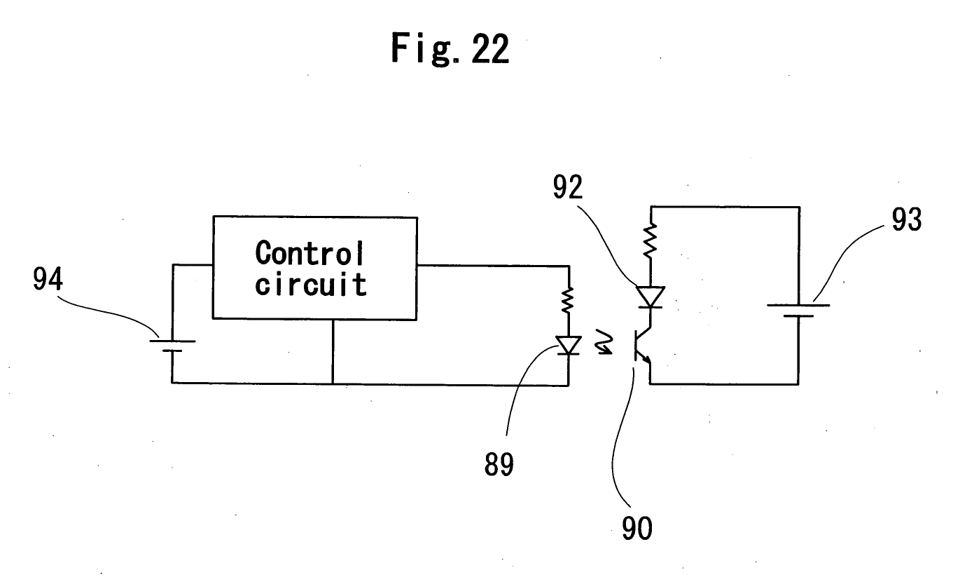

### FIGS. 21 and 22

FIG. 21 is a schematic side view illustrating an electronic wrist watch in accordance with a seventeenth embodiment of the present invention and showing the internal configuration of the electronic wrist watch, which is viewed from the side in a three o'clock direction. FIG. 22 is a circuit block diagram of the electronic wrist watch shown in FIG. 21.

The electronic wrist watch in accordance with the present embodiment has a configuration basically similar to that of the embodiment shown in FIG. 1. Therefore, elements equivalent to those illustrated in FIG. 1 are numerically numbered similarly and the detailed descriptions of the equivalent elements are omitted.

In the electronic wrist watch in accordance with the present embodiment, the light emitting element 6 is composed of an LED 89 for a signal and is disposed in the body case 1 as shown in FIG. 21.

Moreover, a photo detector 90 is disposed in the band 4 in such a manner that the photo detector 90 faces to the LED 89 for a signal via the opening portion 5 of the body case 1.

The photo detector 90 is mounted on a flexible printed circuit board (FPC) 91 disposed in the band 4. An LED 92 for display and a battery 93 for display are disposed on the flexible printed circuit board (FPC) 91, and are connected to the photo detector 90 by a wiring pattern.

Consequently, in the present embodiment, the light guide means 7 is composed of the photo detector 90, the flexible printed circuit board (FPC) 91, the LED 92 for display, and the battery 93 for display.

By such a configuration, as shown in FIGS. 21 and 22, a light emitted from the signal LED 89 disposed in the body case 1 is received by the photo detector 90 disposed in the band 4. The photo detector 90 then drives and lights the LED 92 for display. In FIG. 22, a numeral 94 represents a battery for a watch. The battery 94 for a watch is a power supply means for lighting the LED 89 for a signal, and can also be used as a power supply for the electronic wrist watch.

By such a configuration, the battery 94 for a watch and the battery 93 for display are disposed in the body case 1 and the band 4, respectively. Consequently, an exchanged use of power supplies is not required and a connector for connecting elements is not required, thereby simplifying a configuration of a band connection. Moreover, since a dedicated battery is used for display, a battery inside the watch is not consumed and an operation of the watch is not affected. Furthermore, only the LED 92 for display on the band 4 side can be conveniently exchanged.

While the band 4 is worn on a wrist in the above embodiments, the band 4 can also be worn, for instance, on an arm, an ankle, a knee, a finger, a neck, and other living body parts. Moreover, while the apparatus is applied to a human body in the above embodiments, the apparatus can also be applied, for instance, to animals such as a chimpanzee for a health management. While the preferred embodiments of the present invention have been described above, the present invention is not restricted to the above embodiments, and various changes and modifications can be thus made without departing from the scope of the present invention.

While the present invention is applied to an electronic wrist watch as a worn type electronic device in the above embodiments, the present invention is not restricted to the above embodiments. For instance, the present invention can also be applied to various types of worn type electronic devices such as an arm portable telephone, a television, a toy, and an electronic digital assistant of a worn type.

### INDUSTRIAL APPLICABILITY

The worn type electronic device in accordance with the present invention can be applied, for instance, for an electronic wrist watch having a high fashionable property and a high visibility from other persons, since a light emitting means disposed in the body case and a light guide means disposed in the band can make the band luminous. The worn type electronic device in accordance with the present invention does not require a light emitting means disposed in the band and a power supply means for driving the light emitting means, and does not lose a fitting property of the band itself. Consequently, the worn type electronic device in accordance with the present invention is suitable as an electronic wrist watch in which a fitting property to a human body is important in particular.

The biological measuring apparatus provided with the worn type electronic device in accordance with the present invention enables a visibility and a variety in a representation to be improved even in the case in which a size of the body case is limited. Consequently, the biological measuring apparatus in accordance with the present invention is suitable as an apparatus in which compactness and lightness are required like a wrist watch type. In particular, the biological measuring apparatus in accordance with the present invention is suitable as an apparatus for precisely measuring biological information.

1. A worn type electronic device provided with a light emitting means for emitting a light from the worn type electronic device, comprising:
the light emitting means including a light emitting element for emitting a light having a specified wave length and a light guide means for guiding the light emitted from the light emitting element in a specified direction;
the light emitting element disposed in a body of the worn type electronic device, wherein a light emission of the light emitting element is controlled based on the control of the worn type electronic device; and
the light guide means including at least one of a body side light guide means disposed in the body of the worn type electronic device and a band side light guide means disposed in a band,
wherein the body side light guide means is disposed with a specified face made to face to the surface of the body of the worn type electronic device and the light emitted from the light emitting element is emitted from the surface of the body of the worn type electronic device via the body side light guide means; or
wherein the band side light guide means is disposed with a specified face made to face to the surface of the band and the light emitted from the light emitting element is emitted from the surface of the band via the band side light guide means.

2. The worn type electronic device as defined in claim 1, wherein the light guide means is provided with a body side light guide means disposed in the body of the worn type electronic device and a band side light guide means disposed in a band, and the light emitted from the light emitting element is guided to the band side light guide means via the body side light guide means and is emitted from the surface of the band via the band side light guide means.

3. The worn type electronic device as defined in claim 1, wherein the light guide means is provided with a body side light guide means disposed in the body of the worn type electronic device and a band side light guide means disposed in a band, the light emitted from the light emitting element is emitted from the surface of the body of the worn type electronic device via the body side light guide means, and the light emitted from the light emitting element is guided to the band side light guide means via the body side light guide means and is emitted from the surface of the band via the band side light guide means.

4. The worn type electronic device as defined in claim 1, wherein the band side light guide means is disposed on the surface of the band or disposed in the band.

5. The worn type electronic device as defined in claim 1, wherein a plurality of the light emitting elements is disposed in the body of the worn type electronic device and a plurality of the band side light guide means is disposed in the band corresponding to the light emitting elements.

6. The worn type electronic device as defined in claim 1, wherein a width of the band side light guide means becomes narrower as the position on the band side light guide means becomes farther from the body of the worn type electronic device.

7. The worn type electronic device as defined in claim 1, further comprising at least one shading index portion disposed on the surface of the band.

8. The worn type electronic device as defined in claim 1, wherein the surface of the band has a shading function and at least one index opening portion is formed on the surface of the band.

9. The worn type electronic device as defined in claim 1, further comprising at least one shading index portion disposed on the surface of the body side light guide means.

10. The worn type electronic device as defined in claim 1, wherein the surface of the body side light guide means has a shading function and at least one index opening portion is formed on the surface of the body side light guide means.

11. The worn type electronic device as defined in claim 1, further comprising at least one filter on the surface of a light emission side of the light guide means, the filter for enabling only the light that has been emitted from the light emitting element and that has a specified wave length to pass through the filter.

12. The worn type electronic device as defined in claim 11, further comprising at least one index portion on the surface of the filter, the index portion for emitting a light by the light that has been emitted from the light emitting element and passed through the filter and that has a specified wave length.

13. The worn type electronic device as defined in claim 1, wherein the light emitting element is composed of at least one light emitting element selected from an LED light emitting element, an ultraviolet light emitting element, and an electron beam emitting element.

14. The worn type electronic device as defined in claim 1, wherein the band side light guide means is composed of an optical fiber.

15. The worn type electronic device as defined in claim 1, wherein the band side light guide means is composed of a light guide member provided with diffraction gratings on the both faces thereof.

16. The worn type electronic device as defined in claim 1, further comprising:
at least one liquid crystal element disposed between the light emitting element and the band side light guide means, and
a plurality of index deflection members having deflection angles intersecting with each other at right angles on the surface of the band side light guide means,
wherein a plurality of the index deflection members is selected to diffuse a light by varying a voltage to be applied to the liquid crystal element.

17. The worn type electronic device as defined in claim 1, wherein the light guide means is provided with a light storing means for storing a light guided by the light guide means.

18. A biological measuring apparatus provided with the worn type electronic device as defined in claim 1 for measuring biological information of a user, wherein the biological measuring apparatus is at least one of a pulse wave measuring apparatus for measuring a pulse wave, a body temperature measuring apparatus for measuring a body temperature, and a sweat measuring apparatus for measuring a sweating degree, or a measuring apparatus in which said apparatuses are combined, further comprising:
a biological information value calculation means for detecting biological information of the user to obtain specified biological information values from the biological information;
a user information input means for inputting user information of the user;
a comparison operating means for comparing the user information and the biological information values; and
an informing means for informing of support information corresponding to the comparison results obtained by the comparison operating means,
wherein the informing means is provided with a first informing means for informing of support information in the case in which a light emitted from the light emitting element is emitted from at least one of the surface of the body of the worn type electronic device and the surface of the band via at least one of the body side light guide means disposed in the body of the worn type electronic device and the band side light guide means disposed in the band.

19. The biological measuring apparatus as defined in claim 18, wherein the informing means is further provided with a second informing means for informing of support information by using a character or a pictograph.

20. The biological measuring apparatus as defined in claim 18, wherein
the biological measuring apparatus is a body-mounted type exercise training support apparatus having a function of advising a user who exercises oneself;
the biological information value calculation means is provided with a heart rate calculation means for detecting biological information of the user to calculate a heart rate from the biological information;
the comparison operating means is provided with a heart rate comparison operating means for comparing the user information and the heart rate; and
the informing means is provided with a heart rate informing means for informing of support information corresponding to the comparison results obtained by the heart rate comparison operating means.
